(19) United States
(12) Patent Application Publication
(10) Pub. No.: US 2004/0139494 A1
(43) Pub. Date: Jul. 15, 2004

(54) Infection resistant plants and methods for their generation

(76) Inventors: Ronchang Yang, Bateman (AU); Robert Bower, Dunedin (NL); Kirsi Lehto, Kaarina (FI); Teemu Tapani Juntila, Turku (FI); Eija Pehu, Washington, DC (US)

Correspondence Address: FOLEY AND LARDNER, SUITE 500, 3000 K STREET NW, WASHINGTON, DC, 20007, US

(21) Appl. No.: 10/450,412

(22) PCT Filed: Dec. 13, 2001
(86) PCT No.: PCT/AU01/01611

(30) Foreign Application Priority Data
Dec. 15, 2000 (AU) PR 2103

(51) Int. Cl. A01H 1/00; C12N 15/82

(52) U.S. Cl. 800/279; 800/280

(57) ABSTRACT

The invention relates generally to transgenic plants that are resistant to BYDV infection through the expression of modified forms of messenger RNA (mRNA) that encodes a viral replicase. The method comprises the step of transforming a modified replicase nucleic acid molecule into a plant cell, wherein the expression of said replicase nucleic acid molecule results I the expression of a translationally-altered RNA molecule which confers to said plant resistance against infection with BYDV.

This application is based on and claims the benefit of the filing date of U.S. provisional application 60/292,778 filed 21 May 2001, and Australian provisional application PR2103 filed 15 Dec. 2000.

### FIELD OF THE INVENTION

The invention relates generally to transgenic plants that are resistant to viral infection through the expression of modified forms of messenger RNA (mRNA). In particular, the invention relates to the expression of modified forms of mRNA from an isolated replicase gene. More specifically, the replicase gene is a modified form of a replicase gene isolated from barley yellow dwarf virus (BYDV).

The invention further relates to methods of inducing resistance to BYDV, and plants that are transformed with a modified BYDV replicase gene.

### BACKGROUND OF THE INVENTION

One of the most significant problems associated with agriculture is viral-induced crop damage. Plant viruses are capable of infecting many of the agriculturally important crops, and the damage caused by these infections result in significant losses in crop yield each year. These crop losses reduce the economic value of these crops to the grower, and these losses are eventually passed on to the consumer as higher prices.

Past attempts at controlling or preventing viral infection of plants have concentrated upon either cultivating resistant plant lines that exhibit genetic resistance to virus infection, or controlling viral vectors such as insects. However, while these methods have partially succeeded in reducing the incidence of viral infection, there have been a number of major environmental and agricultural impacts. In particular the indiscriminate use of insecticides has resulted in the death of many non-targets, some of which are beneficial species. Moreover, there has been an increase in significant health risks to humans that are allergic to agricultural chemicals.

With the advent of molecular techniques, a number of approaches for combating plant viruses have been developed. The obvious advantage of such approaches is that the use of expensive and indiscriminate insecticides is reduced. However, there is a further advantage in that the means of providing the protection is incorporated into the plant itself, thereby becoming an inheritable trait which is passed on to its progeny.

A number of molecular approaches have been examined to date including:

1). Transforming susceptible plant species with chimeric genes which express transcripts, or proteins that inhibit viral infection;

2). Expression of viral coat protein or coat protein transcripts;

3). Expression of viral replicases in unmodified or modified form;

4). Expression of antisense genes or ribozymes targeting viral genomic RNA or transcripts; and

5). Expression of altered viral transcripts.

For a review, see Fitchen, J. H. et al., Ann. Rev. Microbiol. 47. 739-763 (1993).

Viral resistance is often described as the ability of a plant to either prevent infection, to suppress or retard the multiplication of a virus, or to suppress or retard the development of pathogenic symptoms (Cooper and Jones, 1983). Several different types of viral resistance are recognised, including inhibition of:

1). Establishment of infection;

2). Virus multiplication, or

3). Viral movement.

One known type of protection against viral infection is termed coat protein-mediated resistance, which involves the expression of a plant virus capsid protein. However, even though this type of resistance has proven to be useful in a variety of situations, it is not always the most effective or efficient means of providing viral resistance. U.S. Pat. No. 6,013,864 describes a method of genetically engineering plants, wherein the plant expresses a replicase gene taken from a plant virus. Upon expression of the replicase gene in the plant the infecting virus is unable to become established in the plant. It is thought that the expressed transgene protein interferes with the function of the protein synthesised by the plant when infected by virus. The proper function of the protein is required by the virus for normal rates of replication within the infected plant cells. However, the major problem with this method is that a foreign gene needs to be incorporated into the genome of the plant, and then transcribed and translated at a sufficient level to enable the plant to withstand viral infection. In other words, this process requires the plant to expend a large amount of energy in transcribing and translating a foreign protein, and maintaining this level of expression throughout its life. This potentially impacts on the quality and quantity of the crops produced. In addition, consumers have increasingly expressed concerns about the consumption of viral proteins. Accordingly, alternate means of protecting plants from viral infection are required which do not over burden the plant in expression of the protective mechanism.

One means that has recently been investigated, is the expression of modified viral transcripts. However, the most recent reports have shown that the expression of viral coat protein transcripts, that have been modified to render them incapable of translation, have produced only limited success in protecting plants. Moreover, the reports are limited to the expression of such “untranslatable” viral transcripts in dicotyledonous plants like tobacco (Lindbo, J. A. et al., Mol. Plant-Microbe Int. 5(2): 144-153 (1992); Lindbo, J. A. et al., Virology 189: 725-733 (1992); PCT application publication no. WO93/17098 to Dougherty, W. G. et al. (Sep. 2, 1993); Lindbo, J. A. et al., The Plant Cell 5: 1749-1759 (1993)), tomato spotted wilt virus (Pang, S. et al., Biotechnology 11: 819-824 (1993); DeHaan et al, Bio/Technology 10: 1133-1137 (1992) and potato virus Y (Van der Vlugt, R. A. et al., Plant Mol. Biol. 17: 431-439 (1991).

While the use of “untranslatable” RNA to inhibit viral infection appears to be more useful than protein expression-based systems, the capacity for this method to be used in all types of plants, especially monocotyledonous plants, appears limited. For example, failure of such altered viral transcripts to inhibit viral infection have been reported for tobacco mosaic virus (Powell, P. A. et al., Virology 175: 124-130 (1990) and zucchini yellow mosaic virus (Pang, G. et al., Mol. Plant-Microbe Int. 6(3): 358-367 (1993), a potyvirus similar to tobacco etch virus. Additional unreported failures may also exist, since such negative results are rarely published. Accordingly, there is still a need for a broad-based system of protection.

One of the major crop pests is barley yellow dwarf virus (BYDV). BYDV causes mosaic symptoms and dwarfing of infected plants, ultimately reducing crop yields (Knoke, J. K. et al., pages 235-281 of “Diseases of Cereals & Pulses”, volume 1, ed. by Singh, U. S. et al., pub. by Prentice Hall, Englewood Cliffs, N.J. (1992)). BYDV is prevalent world-wide and has a wide host range in the Poaceae. It is the major viral pathogen of cereal crops (Lister and Ranieri, 1995). Wheat plants infected with BYDV may show symptoms of chlorosis, reduced growth and a decline in yield, while other cereals show symptoms of yellowing and stunting (barley), yellowing, reddening, leaf stiffening, reduced tillering and heading, and numerous sterile florets (oats) (Miller and Rasochova, 1997). The effects of BYDV on yield can be estimated by use of insecticides to control aphids, its obligate vector. It has been estimated that two applications per year of insecticide would increase the yield by 28%, 25% and 20% for oat, wheat and barley, respectively (McKirdy and Jones, 1996). Estimates of crop losses from BYDV in the United States in 1989, based on a hypothetical 5% loss, were corn $US847m, wheat $US387m, barley $US49m, oats $US28m (Hewings and Eastman, 1995). Use of insecticides to control aphid transmission of the virus is expensive and is considered to have negative effects on the environment, and attempts to find useable sources of natural resistance in wheat have not been successful (Anderson et al., 1998; Francki et al., 1997). Accordingly, to date, there has only been limited success in reducing the adverse impact of this virus. Thus there remains a need to identify additional effective means for protecting host plants from BYDV.

BYDV is classified as a member of the luteovirus plant virus group. Luteoviruses are positive-sense, single-stranded RNA viruses. To form a viral particle, the viral RNA is encapsidated by the coat protein to give the characteristic isometric shape typical of viruses in the luteovirus group. BYDV is non-persistently transmitted to cereal crops and wild grass species by aphids (see Hollings, M. and Brunt, A., pages 732-807 of “Handbook of Plant Virus Infection and Comparative Diagnosis”, Ed. by E. Kurstak, pub. by Elsevier/North Holland Biomedical Press, Amsterdam (1981)).

While several full length sequences of BYDV isolates have been identified, the functions of the open reading frames (ORFs) located within these sequences are only partially understood (see Miller et al: 1988a; Miller and Rasochova, Annual Rev. Phytopathol. 1997, 35: 167-90).

The applicant has now surprisingly found a method by which BYDV infections may be reduced or prevented in plants. This method preferably utilises a genetically modified replicase gene from BYDV, although other BYDV genes may also be utilised. Once a plant has been transformed with the modified gene the resulting expression of mRNA produces a cellular response in the plant whereby the transcribed mRNA is selectively degraded. More importantly, the cellular response induced is incapable of discriminating against other mRNA species of similar sequence. Consequently, when a plant, transformed with the modified gene of the invention, is infected with BYDV the mRNA produced as a result of the infection, having a similar sequence to the mRNA expressed by the modified gene, is also selectively degraded, thereby preventing the infection becoming established in the plant.

### SUMMARY OF THE INVENTION

In its most general aspect, the invention disclosed herein provides a method of protecting plants from BYDV infection. The method utilises RNA-mediated gene silencing, wherein the degradation of a predetermined mRNA is provided. The method may use any modified gene from BYDV, provided that it satisfies the criteria of protecting plants from BYDV infection when it is expressed.

Accordingly, in a first aspect, the present invention provides a method for protecting a plant from BYDV infection, comprising the step of introducing a modified nucleic acid molecule into a plant, wherein the expression of said nucleic acid molecule results in expression of translationally-altered RNA molecule which enables the plant to selectively degrade mRNA produced as a result of contact with BYDV.

The nucleic acid molecule may be cDNA, RNA, or a hybrid molecule thereof. It will be clearly understood that the term nucleic acid molecule encompasses a full-length molecule or a biologically active fragment thereof.

Preferably the nucleic acid molecule is a cDNA molecule encoding a replicase. Most preferably the cDNA molecule is substantially that shown in SEQ ID NO:1, but has been modified, either prior to, or during, integration into the plant genome such that upon expression the mRNA produced has an altered conformation from that of the naturally-occurring mRNA. SEQ ID NO.:1 shows nucleotides 1 to 1610 of ORF2 from BYDV which encodes the catalytic domain of the RNA-dependent RNA polymerase.

The nucleic acid molecule may integrate into the host cell genome, or may exist as an extrachromosomal element.

In a further preferred embodiment, the nucleic acid molecule is modified so that a truncated mRNA is produced upon expression so that the inability to produce functional protein is enhanced.

In a second aspect, the present invention provides a transgenic plant, plant material seeds or progeny thereof, comprising a nucleic acid molecule, wherein the expression of said nucleic acid molecule results in expression of translationally-altered RNA molecule which enables the plant to selectively degrade mRNA produced as a result of contact with BYDV.

Preferably, the nucleic acid molecule is a modified BYDV gene.

Preferably, the plant is a monocot. More preferably, the plant is selected from the group consisting of wheat, sorghum, rice, barley, maize, rye, triticale and oat. Most preferably the plant is wheat, and the modified BYDV gene is a replicase gene isolated from BYDV which has been modified so that upon its expression the mRNA produced induces the host plant to selectively degrade mRNA from BYDV.

In a third aspect, the present invention provides a modified BYDV replicase gene. Preferably, the replicase gene has either

a) a nucleotide sequence as shown in SEQ ID NO:1; or

b) a biologically active fragment of the sequence in a); or

c) a nucleic acid molecule which has at least 75% sequence homology to the sequence in a) or b); or

d) a nucleic acid molecule which is capable of hybridizing to the sequence in a) or b) under stringent conditions as herein defined.

In a fourth aspect, the present invention provides a nucleic acid construct comprising a promoter and a modified BYDV replicase gene as herein defined. Preferably the construct is one of those shown in FIGS. 1 to 10. However, it will be appreciated that modified and variant forms of the constructs may be produced in vitro, by means of chemical or enzymatic treatment, or in vivo by means of recombinant DNA technology. Such constructs may differ from those disclosed, for example, by virtue of one or more nucleotide substitutions, deletions or insertions, but substantially retain a biological activity of the construct or nucleic acid molecule of this invention.

### BRIEF DESCRIPTION OF THE FIGURES

FIG. 1 shows a Single Strand Conformation Polymorphism (SSCP) gel of BYDV Replicase.

FIG. 2 shows the map of the cassette of the CoYMV promoter—BYDV-Rep3 gene—nos terminator in the pUC18 vector.

FIG. 3 shows the map of the cassette of the CoYMV promoter—BYDV-Rep5 gene—nos terminator in the pUC18 vector.

FIG. 4 shows the map of the cassette of the CoYMV promoter—BYDV-RepF gene—nos terminator in pUC18 vector.

FIG. 5 shows the map of the cassette of the CoYMV promoter—BYDV-RepW1 gene—nos terminator in the pUC 18 vector.

FIG. 6 shows the map of the cassette of the CoYMV promoter—BYDV-Rep3 plus Gus gene—nos terminator in the pUC18 vector.

FIG. 7 shows the map of the cassette of the CoYMV promoter—BYDV-Rep3 (sense) plus RepF (antisense) gene—nos terminator in the pUC18 vector.

FIG. 8 shows the map of the cassette of the ocs enhancer—CoYMV promoter—BYDV-RepF gene—nos terminator in the pUC18 vector.

FIG. 9 shows the map of the cassette of the ocs enhancer —CoYMV promoter—BYDV-RepW1 gene—nos terminator in pUC18 vector

FIG. 10 shows the map of the cassette of the ocs enhancer—Ubi promoter—Intron—BYDV-RepF gene—nos terminator in pUC 18 vector.

FIG. 11 shows the map of the cassette of the ocs enhancer—Ubi promoter—Intron—BYDV-RepFW1 gene—nos terminator in pUC 18 vector

FIG. 12 shows the PCR products for the 5′ truncated replicase gene (pCYRep3) generated with primers Rep4 and Rep5.

FIG. 13 shows the results of RT-PCR assays for the BYDV-PAV replicase mRNA in wheat plants.

### DEFINITIONS

The description that follows makes use of a number of terms used in recombinant DNA technology. Unless defined otherwise, all technical and scientific terms used herein have the meaning commonly understood by a person skilled in the art to which this invention belongs. The following references provide one of skill with a general definition of many of the terms used in this invention: Singleton, et al., Dictionary of Microbiology and Molecular Biology (2nd ed. 1994); The Cambridge Dictionary of Science and Technology (Walker ed., 1988); The Glossary of Genetics, 5^{th} Ed., Rieger, R., et al. (eds.), Springer Verlag (1991); and Hale & Marham, The Harper Collins Dictionary of Biology (1991). However, in order to provide a clear and consistent understanding of the specification and claims, including the scope given such terms, the following definitions are provided.

The term “cell” can refer to any cell from a plant, including but not limited to, somatic cells, gametes or embryos.

“Embryo” refers to a sporophytic plant before the start of germination. Embryos can be formed by fertilisation of gametes by sexual crossing or by selfing. A “sexual cross” is pollination of one plant by another. “Selfing” is the production of seed by self-pollination, ie., pollen and ovule are from the same plant. The term “backcrossing” refers to crossing a F1 hybrid plant to one of its parents. Typically, backcrossing is used to transfer genes, which confer a simply inherited, highly heritable trait into an inbred line. The inbred line is termed the recurrent parent. The source of the desired trait is the donor parent. After the donor and the recurrent parents have been sexually crossed, F, hybrid plants which possess the desired trait of the donor parent are selected and repeatedly crossed (ie., backcrossed) to the recurrent parent or inbred line.

Embryos can also be formed by “embryo somatogenesis” and “cloning.” Somatic embryogenesis is the direct or indirect production of embryos from either cells, tissues or organs of plants.

Indirect somatic embryogenesis is characterised by growth of a callus and the formation of embryos on the surface of the callus.

Direct somatic embryogenesis is the formation of an asexual embryo from a single cell or group of cells on an explant tissue without an intervening callus phase. Because abnormal plants tend to be derived from a callus, direct somatic embryogenesis is preferred.

The common term, “grain” is the endosperm present in the ovules of a plant.

The phrase “introducing a nucleic acid sequence” refers to introducing nucleic acid sequences by recombinant means, including but not limited to, Agrobacterium-mediated transformation, biolistic methods, electroporation, in planta techniques, and the like. The term “nucleic acids” is synonymous with DNA, RNA, and polynucleotides. Such a plant containing the nucleic acid sequences is referred to here as an R, generation plant. R1 plants may also arise from cloning, sexual crossing or selfing of plants into which the nucleic acids have been introduced.

A “nucleic acid molecule” or “polynucleic acid molecule” refers herein to deoxyribonucleic acid and ribonucleic acid in all their forms, ie., single and double-stranded DNA, cDNA, mRNA, and the like.

A “double-stranded DNA molecule” refers to the polymeric form of deoxyribonucleotides (adenine, guanine, thymine, or cytosine) in its normal, double-stranded helix. This term refers only to the primary and secondary structure of the molecule, and does not limit it to any particular tertiary forms. Thus this term includes double-stranded DNA found, inter alia, in linear DNA molecules (eg., restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (ie., the strand having a sequence homologous to the mRNA).

A DNA sequence “corresponds” to an amino acid sequence if translation of the DNA sequence in accordance with the genetic code yields the amino acid sequence (ie., the DNA sequence “encodes” the amino acid sequence).

One DNA sequence “corresponds” to another DNA sequence if the two sequences encode the same amino acid sequence.

Two DNA sequences are “substantially similar” when at least about 85%, preferably at least about 90%, and most preferably at least about 95%, of the nucleotides match over the defined length of the DNA sequences. Sequences that are substantially similar can be identified in a Southern hybridization experiment, for example under stringent conditions as defined for that particular system. Defining appropriate hybridization conditions is within the skill of the art. See eg., Sambrook et al., DNA Cloning, vols. I, II and III. Nucleic Acid Hybridization. However, ordinarily, “stringent conditions” for hybridization or annealing of nucleic acid molecules are those that (1) employ low ionic strength and high temperature for washing, for example, 0.015M NaCl/0.0015M sodium citrate/0.1% sodium dodecyl sulfate (SDS) at 50° C., or (2) employ during hybridization a denaturing agent such as formamide, for example, 50% (vol/vol) formamide with 0.1% bovine serum albumin/0.1% Ficoll/0.1% polyvinylpyrrolidone/50 mM sodium phosphate buffer at pH 6.5 with 750 mM NaCl, 75 mM sodium citrate at 42° C.

Another example is use of 50% formamide, 5×SSC (0.75M NaCl, 0.075M sodium citrate), 50 mM sodium phosphate (pH 6.8), 0.1% sodium pyrophosphate, 5× Denhardt's solution, sonicated salmon sperm DNA (50 μg/mL), 0.1% SDS, and 10% dextran sulfate at 42° C., with washes at 42° C. in 0.2×SSC and 0.1% SDS.

A “heterologous” region or domain of a DNA construct is an identifiable segment of DNA within a larger DNA molecule that is not found in association with the larger molecule in nature. Thus, when the heterologous region encodes a plant gene, the gene will usually be flanked by DNA that does not flank the plant genomic DNA in the genome of the source organism. Another example of a heterologous region is a construct where the coding sequence itself is not found in nature (eg., a cDNA where the genomic coding sequence contains introns, or synthetic sequences having codons different than the native gene). Allelic variations or naturally-occurring mutational events do not give rise to a heterologous region of DNA as defined herein.

A “coding sequence” is an in-frame sequence of codons that correspond to or encode a protein or peptide sequence. Two coding sequences correspond to each other if the sequences or their complementary sequences encode the same amino acid sequences. A coding sequence in association with appropriate regulatory sequences may be transcribed and translated into a polypeptide in vivo. A polyadenylation signal and transcription termination sequence will usually be located 3′ to the coding sequence.

“Transgenic plants” are plants into which a nucleic acid has been introduced through recombinant techniques, eg., nucleic acid-containing vectors. A “vector” is a nucleic acid composition which can transduce, transform or infect a cell, thereby causing the cell to express vector-encoded nucleic acids and, optionally, proteins other than those native to the cell, or in a manner not native to the cell. A vector includes a nucleic acid (ordinarily RNA or DNA) to be expressed by the cell. A vector optionally includes materials to aid in achieving entry of the nucleic acid into the cell, such as a retroviral particle, liposome, protein coating or the like. Vectors contain nucleic acid sequences that allow their propagation and selection in bacteria or other non-plant organisms. For a description of vectors and molecular biology techniques, see Current Protocols in Molecular Biology, Ausubel, et al., (eds.), Current Protocols, a joint venture between Greene Publishing Associates, Inc. and John Wiley & Sons, Inc., (through and including the 1998 Supplement) (Ausubel).

“Plasmids” are one type of vector which comprises DNA that is capable of replicating within a plant cell, either extra-chromosomally or as part of the plant cell chromosome(s), and are designated by a lower case “p” preceded and/or followed by capital letters and/or numbers. The starting plasmids herein are commercially available, are publicly available on an unrestricted basis, or can be constructed from such available plasmids by methods disclosed herein and/or in accordance with published procedures. In certain instances, as will be apparent to the ordinarily skilled worker, other plasmids known in the art may be used interchangeably with plasmids described herein.

The phrase “expression cassette” refers to a nucleic acid sequence within a vector, which is to be transcribed, and a control sequence to direct the expression. The term “control sequences” refers to DNA sequences necessary for the expression of an operably linked nucleotide coding sequence in a particular host cell. The control sequences suitable for expression in prokaryotes, for example, include origins of replication, promoters, ribosome binding sites, and transcription termination sites. The control sequences that are suitable for expression in eukaryotes, for example, include origins of replication, promoters, ribosome-binding sites, polyadenylation signals, and enhancers. One of the most important control sequences is the promoter.

A “promoter” is an array of nucleic acid control sequences that direct transcription of a nucleic acid. As used herein, a promoter includes necessary nucleic acid sequences near the start site of transcription, such as, in the case of a polymerase II type promoter, a TATA element.

A promoter also optionally includes distal enhancer or repressor elements, which can be located as much as several thousand base pairs from the start site of transcription. The promoter can either be homologous, ie., occurring naturally to direct the expression of the desired nucleic acid or heterologous, ie., occurring naturally to direct the expression of a nucleic acid derived from a gene other than the desired nucleic acid. Fusion genes with heterologous promoter sequences are desirable, e.g., for regulating expression of encoded proteins. A “constitutive” promoter is a promoter that is active in a selected organism under most environmental and developmental conditions. An “inducible” promoter is a promoter that is under environmental or developmental regulation in a selected organism.

Examples include promoters from plant viruses such as the 35S promoter from cauliflower mosaic virus (CaMV), as described in Odell et al., (1985), Nature, 313:810-812, and promoters from genes such as rice actin (McElroy et al., (1990), Plant Cell, 163-171); ubiquitin (Christensen et al., (1992), Plant Mol. Biol. 12:619-632; and Christensen, et al., (1992), Plant Mol. Biol. 18:675-689); pEMU (Last, et al., (1991), Theor. Appl. Genet 81:581-588); MAS (Velten et al., (1984), EMBO J. 3:2723-2730); and maize H3 histone (Lepetit et al., (1992), Mol. Gen. Genet. 231:276-285; and Atanassvoa et al., (1992), Plant Journal 2(3):291-300).

Additional regulatory elements that may be connected to the viral nucleic acid sequence for expression in plant cells include terminators, polyadenylation sequences, and nucleic acid sequences encoding signal peptides that permit localisation within a plant cell or secretion of the protein from the cell. Such regulatory elements and methods for adding or exchanging these elements with the regulatory elements of the replicase gene are known, and include, but are not limited to, 3′ termination and/or polyadenylation regions such as those of the Agrobacterium tumefaciens nopaline synthase (nos) gene (Bevan et al., (1983), Nucl. Acids Res. 12:369-385); the potato proteinase inhibitor II (PINII) gene (Keil, et al., (1986), Nucl. Acids Res. 14:5641-5650; and An et al., (1989), Plant Cell 1:115-122); and the CaMV 19S gene (Mogen et al., (1990), Plant Cell 2:1261-1272).

Plant signal sequences, including, but not limited to, signal-peptide encoding DNA/RNA sequences which target proteins to the extracellular matrix of the plant cell (Dratewka-Kos, et al., (1989), J. Biol. Chem. 264:4896-4900), the Nicotiana plumbaginifolia extension gene (DeLoose, et al., (1991), Gene 99:95-100), signal peptides which target proteins to the vacuole like the sweet potato sporamin gene (Matsuka, et al., (1991), PNAS 88:834) and the barley lectin gene (Wilkins, et al., (1990), Plant Cell, 2:301-313), signal peptides which cause proteins to be secreted such as that of PRIb (Lind, et al., (1992), Plant Mol. Biol. 18:47-53), or the barley alpha amylase (BAA) (Rahmatullah, et al. “Nucleotide and predicted amino acid sequences of two different genes for high-pI alpha-amylases from barley.” Plant Mol. Biol. 12:119 (1989) and hereby incorporated by reference), or from the present invention the signal peptide from the ESP1 or BEST1 gene, or signal peptides which target proteins to the plastids such as that of rapeseed enoyl-Acp reductase (Verwaert, et al., (1994), Plant Mol. Biol. 26:189-202) are useful in the invention.

For the purposes of the present invention, the promoter sequence is bounded at its 3′ terminus by the translation start codon of a coding sequence, and extends upstream to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase.

An “exogenous” element is one that is foreign to the host cell, or is homologous to the host cell but in a position within the host cell in which the element is ordinarily not found.

“Digestion” of DNA refers to the catalytic cleavage of DNA with an enzyme that acts only at certain locations in the DNA. Such enzymes are called restriction enzymes or restriction endonucleases, and the sites within DNA where such enzymes cleave are called restriction sites. If there are multiple restriction sites within the DNA, digestion will produce two or more linearized DNA fragments (restriction fragments). The various restriction enzymes used herein are commercially available, and their reaction conditions, cofactors, and other requirements as established by the enzyme manufacturers are used. Restriction enzymes are commonly designated by abbreviations composed of a capital letter followed by other letters representing the microorganism from which each restriction enzyme originally was obtained and then a number designating the particular enzyme. In general, about 1 μg of DNA is digested with about 1-2 units of enzyme in about 20 μl of buffer solution. Appropriate buffers and substrate amounts for particular restriction enzymes are specified by the manufacturer, and/or are well known in the art.

“Recovery” or “isolation” of a given fragment of DNA from a restriction digest typically is accomplished by separating the digestion products, which are referred to as “restriction fragments,” on a polyacrylamide or agarose gel by electrophoresis, identifying the fragment of interest on the basis of its mobility relative to that of marker DNA fragments of known molecular weight, excising the portion of the gel that contains the desired fragment, and separating the DNA from the gel, for example by electroelution.

“Ligation” refers to the process of forming phosphodiester bonds between two double-stranded DNA fragments. Unless otherwise specified, ligation is accomplished using known buffers and conditions with 10 units of T4 DNA ligase per 0.5 μg of approximately equimolar amounts of the DNA fragments to be ligated.

“Oligonucleotides” are short-length, single- or double-stranded polydeoxynucleotides that are chemically synthesized by known methods (involving, for example, triester, phosphoramidite, or phosphonate chemistry), such as described by Engels, et al., Agnew. Chem. Int. Ed. Engl. 28:716-734 (1989). They are then purified, for example, by polyacrylamide gel electrophoresis.

“Polymerase chain reaction,” or “PCR,” as used herein generally refers to a method for amplification of a desired nucleotide sequence in vitro, as described in U.S. Pat. No. 4,683,195. In general, the PCR method involves repeated cycles of primer extension synthesis, using two oligonucleotide primers capable of hybridizing preferentially to a template nucleic acid. Typically, the primers used in the PCR method will be complementary to nucleotide sequences within the template at both ends of or flanking the nucleotide sequence to be amplified, although primers complementary to the nucleotide sequence to be amplified also may be used. Wang, et al., in PCR Protocols, pp.70-75 (Academic Press, 1990); Ochman, et al., in PCR Protocols, pp. 219-227; Triglia, et al., Nucl. Acids Res. 16:8186 (1988).

“PCR cloning” refers to the use of the PCR method to amplify a specific desired nucleotide sequence that is present amongst the nucleic acids from a suitable cell or tissue source, including total genomic DNA and cDNA transcribed from total cellular RNA. Frohman, et al., Proc. Nat. Acad. Sci. USA 85:8998-9002 (1988); Saiki, et al., Science 239:487-492 (1988); Mullis, et al., Meth. Enzymol. 155:335-350 (1987).

For purposes of describing the present invention, the term “modified” refers to an introduced alteration to a nucleic acid molecule such that, upon transcription, a “translationally-altered RNA” is produced. The term “translationally-altered RNA” is used to refer to a modified form of a naturally-occurring messenger RNA sequence which cannot be completely translated compared to the unmodified, naturally-occurring form. A translationally altered RNA may be incapable of being translated at all or it may be capable of being partially translated into an attenuated peptide corresponding to a portion of the peptide encoded by the naturally occurring messenger RNA sequence from which the translationally altered RNA is derived.

The coding sequence for a naturally-occurring viral RNA sequence may be modified to encode a translationally altered RNA, for example, by removing its ATG initiation codon or by utilising a portion which does not include the initiation codon. Other means for translationally altering a naturally-occurring viral RNA molecule include introducing one or more premature stop codons and/or interrupting the reading frame.

The phrase “operably encodes” refers to the functional linkage between a promoter and a second nucleic acid sequence, wherein the promoter sequence initiates transcription of RNA corresponding to the second sequence.

The term “progeny” refers to the descendants of a particular plant (self-cross) or pair of plants (crossed or backcrossed). The descendants can be of the F1, the Fez, or any subsequent generation.

Typically, the parents are the pollen donor and the ovule donor which are crossed to make the progeny plant of this invention.

Parents also refer to F1 parents of a hybrid plant of this invention (the F2 plants). Finally, parents refer to a recurrent parent which is backcrossed to hybrid plants of this invention to produce another hybrid plant of this invention.

The phrase “producing a transgenic plant” refers to producing a plant of this invention. The plant is generated through recombinant techniques, ie., cloning, somatic embryogenesis or any other technique used by those of skill to produce plants.

The common names of plants used throughout this disclosure refer to varieties of plants of the following genera:

| Common Name | Genera |
|---|---|
| Wheat (soft, hard and | Triticum |
| durum varieties) | |
| Sorghum | Sorghum |
| Rice | Oryza |
| Barley | Hordeum |
| Maize or corn | Zea |
| Rye | Secale |
| Triticale | Triticale |
| Oat | Avena |

“Integration” of the DNA may be effected using non-homologous recombination following mass transfer of DNA into the cells using microinjection, biolistics, electroporation or lipofection. Alternative methods such as homologous recombination, and or restriction enzyme mediated integration (REMI) or transposons are also encompassed, and may be considered to be improved integration methods.

A “clone” is a population of cells derived from a single cell or common ancestor by mitosis.

In addition to the replicase found in BYDV, the term replicase, for purposes of this invention, also refers to replicase homologs. Homologs refers to proteins having a homologous function. Homologs also refer to nucleic acid sequence or amino acid sequence homologs.

“Nucleic acid sequence homologs” refers to deoxyribonucleotides or ribonucleotides and polymers thereof in either single-or double-stranded form containing known analogues of natural nucleotides, which have similar binding properties as the reference nucleic acid and are metabolised in a manner similar to naturally occurring nucleotides.

Unless otherwise indicated, a particular nucleic acid sequence also implicitly encompasses conservatively modified variants thereof (eg., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer, et al., Nucleic Acid Res. 19: 5081 (1991); Ohtsuka, et al., J. Biol. Chem. 260: 2605-2608 (1985); and Rossolini, et al., Mol. Cell. Probes 8: 91-98 (1994)). The term “nucleic acid” is used interchangeably with gene, cDNA, and mRNA encoded by a gene.

The term “amino acid sequence homology” refers to a protein with a similar amino acid sequence. One of skill will realise that the critical amino acid sequence is within a functional domain of a protein. Thus, it may be possible for a homologous protein to have less than 40% homology over the length of the amino acid sequence, but greater than 90% homology in one functional domain. In addition to naturally occurring amino acids, homologs also encompass proteins in which one or more amino acid residue is an artificial chemical analog of a corresponding naturally occurring amino acid, as well as to naturally occurring proteins.

Amino acids may be referred to herein by either their commonly known three letter symbols or by the one-letter symbols recommended by the IUPAC-IUB Biochemical Nomenclature Commission.

Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids that encode identical or essentially identical amino acid sequences, or where the nucleic acid does not encode an amino acid sequence, to essentially identical sequences.

Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein, which encodes a polypeptide, also describes every possible silent variation of the nucleic acid. One of skill will recognise that each codon in a nucleic acid (except AUG, which is ordinarily the only codon for methionine) can be modified to yield a functionally identical molecule. Accordingly, each silent variation of a nucleic acid, which encodes a polypeptide, is implicit in each described sequence.

As to amino acid sequences, one of skill will recognise that individual substitutions, deletions or additions to a nucleic acid, peptide, polypeptide, or protein sequence that alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

(see, e.g., Creighton, PROTEINS (1984)).

As used herein, the terms “transformation” and “transfection” refer to the process of introducing a desired nucleic acid, such a plasmid or an expression vector, into a plant cells, either in culture or in the organs of a plant by a variety of techniques used by molecular biologists. Accordingly, a cell has been “transformed” by exogenous DNA when such exogenous DNA has been introduced inside the cell wall. Exogenous DNA may or may not be integrated (covalently linked) to chromosomal DNA making up the genome of the cell. In prokaryotes and yeast, for example, the exogenous DNA may be maintained on an episomal element such as a plasmid. With respect to eukaryotic cells, a stably transformed cell is one in which the exogenous DNA is inherited by daughter cells through chromosome replication. This stability is demonstrated by the ability of the eukaryotic cell to establish cell lines or clones comprised of a population of daughter cells containing the exogenous DNA.

Numerous methods for introducing foreign genes into plants are known and can be used to insert a modified nucleic acid into a plant host, including biological and physical plant transformation protocols. See, for example, Miki et al., (1993), “Procedure for Introducing Foreign DNA into Plants”, In: Methods in Plant Molecular Biology and Biotechnology, Glick and Thompson, eds., CRC Press, Inc., Boca Raton, pages 67-88. The methods chosen vary with the host plant, and include chemical transfection methods such as calcium phosphate, microorganism-mediated gene transfer such as Agrobacterium (Horsch, et al., (1985), Science 227:1229-31), electroporation, micro-injection, and biolistic bombardment.

Expression cassettes and vectors and in vitro culture methods for plant cell or tissue transformation and regeneration of plants are known and available. See, for example, Gruber, et al., (1993), “Vectors for Plant Transformation” In: Methods in Plant Molecular Biology and Biotechnology, Glick and Thompson, eds. CRC Press, Inc., Boca Raton, pages 89-119.

The most widely utilised method for introducing an expression vector into plants is based on the natural transformation system of Agrobacterium. A. tumefaciens and A. rhizogenes are plant pathogenic soil bacteria which genetically transform plant cells. The Ti and R1 plasmids of A. tumefaciens and A. rhizogenes, respectfully, carry genes responsible for genetic transformation of plants. See, for example, Kado, (1991), Crit. Rev. Plant Sci. 10: 1. Descriptions of the Agrobacterium vector systems and methods for Agrobacterium-mediated gene transfer are provided in Gruber et al., supra; Miki, et al., supra; and Moloney et al., (1989), Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into host organisms show fidelity in gene expression with respect to tissue/organ specificity of the original coding sequence. See, eg., Benfey, P. N., and Chua, N. H. (1989) Science 244: 174-181. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion, or via a binary system where the vir gene is present on a separate vector. Such systems, vectors for use therein, and methods of transforming plant cells are described in U.S. Pat. No. 4,658,082; U.S. application Ser. No. 913,914, filed Oct. 1, 1986, as referenced in U.S. Pat. No. 5,262,306, issued Nov. 16, 1993 to Robeson, et al.; and Simpson, R. B., et al. (1986) Plant Mol. Biol. 6: 403-415 (also referenced in the '306 patent); all incorporated by reference in their entirety.

Once constructed, these plasmids can be placed into A. rhizogenes or A. tumefaciens and these vectors used to transform cells of plant species, which are ordinarily susceptible to BYDV infection. Several other transgenic plants are also contemplated by the present invention including but not limited to soybean, corn, sorghum, alfalfa, rice, clover, cabbage, banana, coffee, celery, tobacco, cowpea, cotton, melon and pepper. The selection of either A. tumefaciens or A. rhizogenes will depend on the plant being transformed thereby. In general A. tumefaciens is the preferred organism for transformation. Most dicotyledons, some gymnosperms, and a few monocotyledons (eg. certain members of the Liliales and Arales) are susceptible to infection with A. tumefaciens. A. rhizogenes also has a wide host range, embracing most dicots and some gymnosperms, which includes members of the Leguminosae, Compositae and Chenopodiaceae. Alternative techniques, which have proven to be effective in genetically transforming plants, include particle bombardment and electroporation. See eg. Rhodes, C. A., et al. (1988) Science 240: 204-207; Shigekawa, K. and Dower, W. J. (1988) Bio/Techniques 6: 742-751; Sanford, J. C., et al. (1987) Particulate Science & Technology 5:27-37; and McCabe, D. E. (1988) Bio/Technology 6:923-926.

Once transformed, these cells can be used to regenerate transgenic plants, capable of withstanding BYDV infection. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors and cultured under conditions, which promote plant regeneration. Roots or shoots transformed by inoculation of plant tissue with A. rhizogenes or A. tumefaciens, containing the gene coding for the BYDV resistance, can be used as a source of plant tissue to regenerate BYDV-resistant transgenic plants, either via somatic embryogenesis or organogenesis. Examples of such methods for regenerating plant tissue are disclosed in Shahin, E. A. (1985) Theor. Appl. Genet. 69:235-240; U.S. Pat. No. 4,658,082; Simpson, R. B., et al. (1986) Plant Mol. Biol 6: 403-415; and U.S. patent applications Ser. Nos. 913,913 and 913,914, both filed Oct. 1, 1986, as referenced in U.S. Pat. No. 5,262,306, issued Nov. 16, 1993 to Robeson, et al.; the entire disclosures therein incorporated herein by reference.

Despite the fact that the host range for Agrobacterium-mediated transformation is broad, some major cereal crop species and gymnosperms have generally been recalcitrant to this mode of gene transfer, even though some success has recently been achieved in rice (Hiei et al., (1994), The Plant Journal 6:271-282). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 mu.m. The expression vector is introduced into plant tissues with a biolistic device that accelerates the microprojectiles to speeds of 300 to 600 m/s which is sufficient to penetrate the plant cell walls and membranes. (Sanford et al., (1987), Part. Sci. Technol. 5:27; Sanford, 1988, Trends Biotech 6:299; Sanford, (1990), Physiol. Plant 79:206; Klein et al., (1992), Biotechnology 10:268).

Another method for physical delivery of DNA to plants is sonication of target cells as described in Zang et al., (1991), Bio/Technology 9:996. Alternatively, liposome or spheroplast fusions have been used to introduce expression vectors into plants. See, for example, Deshayes et al., (1985), EMBO J. 4:2731; and Christou et al., (1987), PNAS USA 84:3962. Direct uptake of DNA into protoplasts, using CaCl₂ precipitation, polyvinyl alcohol or poly-L-ornithine, have also been reported. See, for example, Hain et al., (1985), Mol. Gen. Genet. 199:161; and Draper et al., (1982), Plant Cell Physiol. 23:451.

Electroporation of protoplasts and whole cells and tissues has also been described. See, for example, Donn et al., (1990), In: Abstracts of the VII^{th} Int'l. Congress on Plant Cell and Tissue Culture IAPTC, A2-38, page 53; D'Halluin et al., (1992), Plant Cell 4:1495-1505; and Spencer et al., (1994), Plant Mol. Biol. 24:51-61.

Alternatively, the DNA constructs are combined with suitable T-DNA flanking regions and introduced into a conventional Agrobacterium tumefaciens host vector. The virulence functions of the Agrobacterium tumefaciens host directs the insertion of the construct and adjacent marker into the plant cell DNA when the cell is infected by the bacteria.

Microinjection techniques are known in the art and well described in the scientific and patent literature. The introduction of DNA constructs using polyethylene glycol precipitation is described in Paszkowski, et al., EMBO J. 3: 2717 (1984). Electroporation techniques are described in Fromm, et al., Proc. Nat'l. Acad. Sci. USA 82: 5824 (1985). Ballistic transformation techniques are described in Klein, et al., Nature 327: 70-73 (1987).

Agrobacterium tumefaciens-mediated transformation techniques, including disarming and use of binary vectors, are also well described in the scientific literature. See, for example Horsch, et al., Science 233: 496-498 (1984), and Fraley, et al., Proc. Nat'l. Acad. Sci. USA 80: 4803 (1983).

One preferred method of transforming plants of the invention is microprojectile bombardment. In this method target tissues are treated with osmoticum. Then modified BYDV gene DNA is precipitated, and coated on to tungsten or gold microparticles. The microparticles are then loaded into microprojectile or biolistic device and the treated cells are bombarded (Bower et al., 1996).

### DETAILED DESCRIPTION OF THE INVENTION

The basis of the present invention is the discovery that reduced susceptibility to infection by BYDV may be conferred upon a plant, especially a monocotyledonous plant, by producing in the plant a modified RNA molecule corresponding in sequence to a plus-sense or messenger RNA molecule of the target BYDV.

The practice of the present invention employs, unless otherwise indicated, conventional molecular biology, microbiology, and recombinant DNA techniques within the skill of the art. Such techniques are well known to the skilled worker, and are explained fully in the literature. See, eg., Maniatis, Fritsch & Sambrook, “Molecular Cloning: A Laboratory Manual” (1982); “DNA Cloning: A Practical Approach,” Volumes I and II (D. N. Glover, Ed., 1985); “Oligonucleotide Synthesis” (M. J. Gait, Ed., 1984); “Nucleic Acid Hybridization” (B. D. Hames & S. J. Higgins, eds., 1985); “Transcription and Translation” (B. D. Hames & S. J. Higgins, eds., 1984); B. Perbal, “A Practical Guide to Molecular Cloning” (1984), and Sambrook, et al., “Molecular Cloning: a Laboratory Manual” 12^{th} edition (1989).

Generally, the nomenclature and the laboratory procedures in plant maintenance and breeding as well as recombinant DNA technology described below are those well known and commonly employed in the art.

The preferred approach for producing the translationally-altered RNA molecule in a plant is by introducing a chimeric gene or modified gene sequence designed to express this molecule in the cells of the plant. Such a chimeric gene may consist of at least two components, a promoter and a coding sequence that is operably linked to the promoter.

The promoter component may be any promoter that is capable of regulating or directing the expression of an operably linked gene in the targeted monocotyledonous plant. Such promoters are well known in the art. For example, a constitutive plant promoter fragment may be employed which will direct expression of the viral sequence in all tissues of a plant. Such promoters are active under most environmental conditions and states of development or cell differentiation.

Examples of constitutive promoters include the cauliflower mosaic virus (CaMV) 35S transcription initiation region, the 1′-or 2′-promoter derived from T-DNA of Agrobacterium tumafaciens, and other transcription initiation regions from various plant genes known to those of skill.

Alternatively, the plant promoter may be under environmental control. Such promoters are referred to here as “inducible” promoters. Examples of environmental conditions that may effect transcription by inducible promoters include pathogen attack, anaerobic conditions, or the presence of light.

Preferably, a promoter that is capable of directing strong expression is used. Such promoters include, but are not limited to, the maize ubiquitin promoter described in Christensen and Quail (1996), the rice actin promoter as described in McElroy D, Blowers A D Jenes B and Wu R (1991), the commelina mosaic virus promoter as described in Medberry S L, Lockhart B EL and Olszewskine (1992).

The vector comprising the sequences from the BYDV sequence will also typically comprise a marker gene which confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosluforon, or phosphinothricin (the active ingredient in bialaphos and Basta).

The coding sequence component comprises a modified nucleic acid sequence which, when transcribed, produces a translationally-altered RNA molecule corresponding to a target viral sequence. The target viral sequence is a mRNA molecule of the target virus, or a portion thereof. Since the target viral sequence is naturally translatable when a translation initiation codon is present, it is modified so as to render it untranslatable. For any given target viral sequence, the skilled artisan will be able to determine various modifications which could be made to render the resulting RNA molecule untranslatable.

While the applicant does not wish to be bound by any particular theory they postulate that the RNA-mediated gene silencing method disclosed herein results from plants being “induced”, in an immune response-like process, to recognise specific mRNAs including those produced as a result of contact with an infecting virus. The introduced BYDV gene encodes a mRNA that is conformationally altered compared to mRNA encoded by the unmodified gene. The plant recognises that this conformationally altered form is “foreign” and a cellular response is induced which selectively degrades that mRNA species. Once induced, the mRNA mediated gene silencing is active against mRNAs with a similar sequence, rather than a conformational identification. Consequently, when an infecting BYDV transcribes its unmodified gene the resulting mRNA of high sequence homology is degraded before it is translated, thereby stopping the infection from progressing.

A further embodiment of the present invention incorporates methods into the above process to ensure that translation of functional protein is reduced to a minimum. This further method involves introducing mutations so that “translationally-altered RNA” is produced as defined herein.

Translation of an mRNA molecule in a plant cell generally requires the presence of an initiation AUG codon followed by an uninterrupted string of amino acid codons ending with a translational stop codon, which may be either UAA, UAG or UGA. A DNA molecule encoding a translatable mRNA molecule may be modified to encode a translationally altered RNA, for instance, by either removing the initiation ATG codon, interrupting the reading frame, adding premature stop codons, or by a combination of these modifications.

Introduction of one or more premature stop codons (encoded by DNA codons TAA, TAG or TGA) in a target viral sequence-may be accomplished by adding or deleting nucleotides or by modifying existing nucleotides using standard techniques such as site directed mutagenesis, or mutagenesis by PCR. Adding or deleting nucleotides may have the additional benefit of interrupting the reading frame, which also has the effect of translationally altering the RNA molecule. While the addition of a premature stop codon anywhere along the length of the target viral sequence will render it translationally altered as that term is used herein to describe the invention, it is preferable to introduce such stop codons near the 5′ end of the target viral mRNA so that any attenuated peptides which may be produced via partial translation are 20 amino acids or less in length.

The reading frame of a target viral sequence may be interrupted by the addition or deletion of nucleotides in the DNA coding sequence. As with the addition of premature stop codons, it is preferable to interrupt the reading frame near the 5′ end of the target viral RNA so that any attenuated peptides corresponding to a portion of the peptide encoded by the target viral RNA which may be produced via partial translation are 20 amino acids or less in length.

Another way to translationally alter the target viral sequence is to remove the translation initiation codon, which will be an ATG. This may be accomplished simply by choosing a target viral sequence which does not include the translation initiation codon. In some cases truncation of significant 5′ portions, of the gene may also be used for this purpose. Alternatively, this may be accomplished by disrupting the ATG codon either by adding, deleting or modifying nucleotides within this codon using standard techniques.

Any mRNA molecule produced by the BYDV, or any portion of such a molecule, may be used as the target sequence. The target sequence is preferably at least 120 nucleotides in length, more preferably at least 250 nucleotides in length, and most preferably at least 500 nucleotides in length.

The target sequence of the present invention may correspond to the coding sequence for any viral protein, such as a viral coat protein replicase, proteinase, inclusion body protein, helicase, 6K protein and VPg. Such sequences are well known for several monocotyledonous viruses including, but not limited to, MDW, Sugarcane mosaic virus (partial sequence; see Frenkel, M. J. et al. J. Gen. ViroL. 72:237-242, (1991)), Johnson grass mosaic virus (partial sequence) (see Gough, K. H. et al., J. Gen. Virol. 68:297-304, (1987), maize chlorotic mottle virus (see Nutter, R. C. et al. Nucleic Acids Research 17:3163-3177, (1989)), maize chlorotic dwarf virus (see International Patent Application no. PCT/US94/03028 published Sep. 29, 1994 as WO94/21796), maize rough dwarf virus (partial sequence) (see Marzachi, C. et al. Virology 180:518-526, (1991)), maize stripe virus (partial sequence) (see Huiet, L. et al. Virology 182:47-53, (1991); Huiet, L. et al. J. Gen. Virol. 73:1603-1607, (1992); Huiet, L. et al. GenBank Accession Number L3446, (1993)), maize streak virus (see Mullineaux, P. M. et al EMBO J. 3:3063-3068, (1984)), barley yellow dwarf virus (see Larkins, B. A. et al. J. Gen. Virol. 72:2347-2355, (1991)), and wheat spindle streak virus (partial sequence) (see Sohn, A. et al. Arch. Wrol. 135:279-292, (1994)).

Suitable host plants which may benefit from the production of translationally altered viral RNA include any monocotyledonous species which are susceptible to viral infection, particularly infection by a member of the luteovirus family. In particular, suitable host plants include maize, wheat, sugarcane, oats, barley, rye, rice (Miller and Rasochova, 1997). It will be clearly understood by persons skilled in the art that references like “Diseases of Cereals and Pulses” (Ed. by Singh, U. S. et al., Prentice Hall, Englewood Cliffs, N.J. (1992)) and Lister and Raineri (1995), identify a number of crops that can act as a viral host. In addition, BYDV infects many species of annual and perennial grass species including pasture species http:www.biology.anu.edu.au/Groups/MES/vide/descr062.

Accordingly, the applicant believes that there is a real expectation that the approaches described herein will be effective in a range of plant species. In particular, the applicant considers that as the replicase gene, as well as others, are required by BYDV to establish an infection and are common to all isolates of BYDV, the usefulness of these as targets for RNA-mediated gene silencing targets is high.

In a preferred embodiment, the target viral sequence used is a coding sequence that is identical or highly homologous among two or more monocotyledonous viruses or virus strains. Expression of translationally altered RNA in a monocotyledonous plant based on such a shared sequence is contemplated to inhibit infection by any of the viruses which produce a mRNA having homology with the target viral sequence.

The isolated BYDV genomic sequences taught by the present invention are particularly useful for the development of viral resistance in susceptible host plants. With the information provided by the present invention, several approaches for inhibiting plant virus infection in susceptible plant hosts which involve expressing in such hosts various inhibitory transcripts or proteins derived from the target virus genome may now be applied to BYDV Use of translationally altered RNA to confer monocotyledonous virus resistance as described herein above may now be applied to BYDV, as demonstrated by Example 4.

The DNA constructs described above may be introduced into the genome of the desired plant host by a variety of conventional techniques as discussed above. However, other techniques for transforming a wide variety of higher plant species are well known and described in the technical and scientific literature. See, for example, Weising, et al., Ann. Rev. Genet. 22: 421-477 (1988).

The DNA construct may be introduced directly into the genomic DNA of the plant cell using techniques such as biolistic methods, electroporation, PEG poration, and microinjection of plant cell protoplasts or embryogenic callus. Alternatively, the DNA constructs may be combined with suitable T-DNA flanking regions and introduced using an A. tumefaciens or A. rhizogenes vector. Particle bombardment techniques are described in Klein, et al., Nature 327: 70-73 (1987). A particularly preferred method of transforming wheat and other cereals is the bombardment of calli derived from immature embryos as described by Weeks, et al., Plant Physiol. 102: 1077-1084 (i993).

Microinjection techniques are known in the art and well described in the scientific and patent literature. The introduction of DNA constructs using polyethylene glycol precipitation is described in Paszkowski, et al., EMBO J. 3: 27172722 (1984). Electroporation techniques are described in Fromm, et al., Proc. Nat'l Acad. Sci. USA 82: 5824 (1985).

Agrobacterium tumefaciens-meditated transformation techniques are also well described in the scientific literature. See, for example Horsch, et al., Science 233: 496-498 (1984), and Fraley, et al. Proc. Nat'l Acad. Sci. USA 80: 4803 (1983).

Although Agrobacterium is useful primarily in dicots, certain monocots can be transformed by Agrobacterium. For instance, Agrobacterium transformation of rice is described by Hiei, et al, Plant J. 6: 271-282 (1994); U.S. Pat. No. 5,187,073; U.S. Pat. No. 5,591,616;

Li, et al., Science in China 34: 54 (1991); and Raineri, et al., Bio/Technology 8: 33 (1990). Xu, et al., Chinese J. Bot. 2: 81 (1990) transformed maize, barley, triticale and asparagus by Agrobacterium infection.

The present invention is particularly useful in wheat and other cereals. A number of methods of transforming cereals have been described in the literature. For instance, transformation of rice is described by Toriyama, et al., Bio/Technology 6: 10721074 (1988), Zhang, et al., Theor. Appl. Gen. 76: 835-840 (1988), and Shimamoto, et al., Nature 338: 274-276 (1989). Transgenic maize regenerants have been described by Fromm, et al., Bio/Technology 8: 833-839 (1990) and Gordon-Kamm, et al., Plant Cell 2: 603-618 (1990)). Similarly, oats (Sommers, et al., Bio/Technology 10: 1589-1594 (1992)), wheat (Vasil, et al., Bio/Technology 10: 667-674 (1992)); Weeks, et al., Plant Physiol. 102: 1077-1084 (1993)), sorghum (Casas, et al., Proc. Nat'l Acad. Sci. USA 90: 11212-11216 (1993)), rice (Li, et al., Plant Cell Rep. 12: 250-255 (1993)), barley (Yuechun & Lemaux, Plant Physiol. 104: 37-48 (1994)), and rye (Castillo, et al., Bio/Technology 12: 1366-1371 (1994)) have been transformed via bombardment.

Transformed plant cells that are derived by any of the above transformation techniques can be cultured to regenerate a whole plant which possesses the transformed genotype and thus the desired phenotype. Such regeneration techniques rely on manipulation of certain phytohormones in a tissue culture growth medium, typically relying on a biocide and/or herbicide marker which has been introduced together with the modified BYDV nucleic acid sequence. Plant regeneration from cultured protoplasts is described in Evans, et al., Protoplasts Isolation and Culture, Handbook of Plant Cell Culture, Macmillian Publishing Company, New York, pp. 124-176 1983; and Binding, Regeneration of Plants, Plant Protoplasts, CRC Press, Boca Raton, pp. 21-73 1985. Regeneration can also be obtained from plant callus, explants, organs, or parts thereof. Such regeneration techniques are described generally in Klee, et al. Ann. Rev. of Plant Phys. 38: 467-486 (1987).

One of skill will recognise that after the expression cassette is stably incorporated in transgenic plants and confirmed to be operable, it can be introduced into other plants by sexual crossing. A technique used to transfer a desired phenotype to a breeding population of plants is through backcrossing. However, any of a number of standard breeding techniques can be used, depending upon the species to be crossed.

The vector will also typically contain an ancillary selectable marker gene by which transformed plant cells can be identified in culture. Usually, the marker gene will encode antibiotic resistance. Other ancillary DNA sequences encoding additional functions may also be present in the vector. For instance, in the case of Agrobacterium transformations, T-DNA sequences will also be included for subsequent transfer to plant chromosomes

After transgenic plants are produced, it is beneficial in selecting subsequent generations to select progeny which contain genetic material which confers a specific beneficial trait, eg., viral resistance. Before selection can begin, however, a genetic map of the desirable genome should be made. Genetic mapping is done by finding polymorphic markers that are genetically linked to each other (in linkage groups) or linked to genes or QTL affecting phenotypic traits of interest. The alignment of markers into linkage groups is useful as a reference for future use of the markers and for accurately positioning genes or QTL relative to the markers. Many of these QTL's have multiple sub-loci and haplotypes across the sub-loci. Each haplotype provides a different allele composition within a locus, thereby expanding the utility of these marker loci to more mapping studies than possible with only two alleles per locus.

The progeny and transgenic plants of this invention can be characterised either genotypically or phenotypically. Genotypic analysis is the determination of the presence or absence of particular genetic material. To determine whether modified viral sequence has been successfully introduced into progeny plants, the parent(s) of the plants of this invention are also analyzed genotypically.

Phenotypic analysis is the determination of the presence or absence of a phenotypic trait. A phenotypic trait is a physical characteristic of a plant determined by the genetic material of the plant in concert with environmental factors. Phenotypic traits can either be simple, eg., Mendelian, or complex, eg., quantitative. Mendelian traits are those conferred upon the hybrid plant by dominant genes.

A quantitative phenotypic trait is one wherein the physical characteristic of the progeny plant is intermediate between the physical trait of the two parents. For purposes of this discussion only, the parents of a transgenic plant are the genome donor and the modified viral sequence donor. An example of a quantitative trait is viral resistance in wheat.

Throughout the specification, the word “comprise” and variations of the word, such as “comprising” and “comprises”, means “including but not limited to” and is not intended to exclude other additives, components, integers or steps.

The invention will now be further described by way of reference only to the following non-limiting examples. It should be understood, however, that the examples following are illustrative only, and should not be taken in any way as a restriction on the generality of the invention described above. Amino acid sequences referred to herein are given in standard single letter code.

### EXAMPLE 1

### Collection and Determination of BYDV Strains

Oat fields, located within a 30 km radius of the town of Turku, Finland, were observed over several months, and samples were collected upon clear visible symptoms of BYDV. In total, 30 field plots were sampled including two plots from the counties of Hiidenvesi (Sample 25) and Nummi-Pusula (Sample 24). Altogether 10-20 plants per plot were collected and stored at −20° C.

A virus-free oat line was maintained as a negative control, and BYDV-PAV isolated by A. W. Miller in Australia, was maintained in oat cv. Heikki, as a positive control. The BYDV-PAV was a good reference isolate as the entire genome has been sequenced (Miller et al. 1988a).

Commercially available ELISA test kits for strains BYDV-PAV, BYDV-RPV and BYDV-MAV (Adgen Agrifood Diagnostics) were used to detect the serotype of the field isolates.

Of the field samples 4 did not react with either of the strain specific ELISA test kit antibodies. Most of the samples were positive only to the BYDV-PAV strain. In all 23 samples were infected with BYDV-PAV. Only one sample was positive only to BYDV-RPV. In two samples both of the virus-strains were present.

### EXAMPLE 2

### Extraction of RNA and Amplification of BYDV Replicase Sequence

Total-RNA was extracted from about 100 mg of leaf material from BYDV infected oat leaves using a Qiagen RNeasy Mini-kit in accordance with the manufacturer's instructions. Extracted RNA for use as a template in cDNA synthesis was stored at −80° C. in 50 μl aliquots.

PCR primers were designed according to the published BYDV-PAV sequence (Miller et al. 1988a, EMBL No XO7653). The primers were designed manually and checked with Oligo v.3.4 and Primers programs (www.wialliamstone.com/primers/) to avoid possible non-specific PCR products and primer pairing. To amplify the replicase sequence primers 39KF and FSR(ORFL) and FSF and POLR (ORF2) were designed. The PCR products of these primers were 602, 1021 and 1608 bp long, respectively, and included the entire coding sequence of the gene. Details of the primers are indicated in Table 1.

TABLE 1

| | | |
|---|---|---|
| 39KF: | CATGTTTTTCGAAATACTAATAGGTGC | (27-mer) |
| FSR: | CTCTAAAAACCCACAGAGTCAAGC | (24-mer) |
| FSF: | CTTGACTCTGTGGGTTTTTAGAG | (23-mer) |
| POLR: | GGTAATTAATATTCGTTTTGTGAGTG | (26-mer) |

cDNA was synthesized by Ready to go You-Primer-First-Strand Beads (Pharmacia Biotech) in accordance with the manufacturer's instructions. Briefly, 10-20 μl of extracted RNA solution was adjusted to 32 μl with DEPC-treated water, and incubated for lomin at 65° C. Following 2 min on ice, reagent beads, and the reverse primer (20-40 pmol/μl) complementary to the virus RNA were added. The sample was incubated for 1 min at room temperature, then mixed carefully, and collected by centrifugation. The sample was then incubated at 37° C. for 1 h.

Following cDNA synthesis PCR amplification was conducted using Ready to Go PCR-Beads (Pharmacia Biotech). In a total reaction volume of 25 μl the following components were mixed: 1.5 U of Taq DNA-polymerase, 5-25 pmol (0.5-1 μl) of forward and reverse primers, 1-10 μl of the template cDNA, and water up to 25 μl. The mixture was vortexed, centrifuged and then amplified in a PTC-200 PCR machine (MJ Research), using the parameters shown in Table 2.

TABLE 2

| | | Temperature^{°} | | |
|---|---|---|---|---|
| Step | | (C.) | Time | |
| 1 | Denaturation | 95 | 5 | min |
| 2 | Denaturation | 95 | 30 | s |
| 3 | Primer annealing | Depends on | 30 | s |
| | | primers | | |
| 4 | Primer extension | 72 | 1 | min |
| 5 | 34 cycles | | | |
| 6 | Primer extension | 72 | 5 | min |

Forward primer = FSF
Reverse primer = POLR
Length of the PCR product = 1608 bp
Annealing temp = 64° C.

PCR products were analyzed on 1% agarose gel, and compared with standards of PstI restricted λ-DNA or 1 kb ladder (Promega). Amplified product was extracted from the agarose gel using QIAEX II product (Qiagen) in accordance with the manufacturer's instructions. Briefly, the desired fragment was excised, weighed and 3 μl of QX 1 buffer was added for each mg of the gel fragment. 10 μl of silica particles were added to the tube, and incubated for 10 min at 50° C. After mixing the sample, the silica and associated DNA was precipitated by 30 s centrifugation. The supernatant was removed and 500 μl of QX 1-buffer was added. The silica was re-suspended, washed twice with PE buffer, then suspended in 20-40 μl of water to elute the DNA. The sample was centrifuged for 30 s and the supernatant transferred to a clean tube.

### EXAMPLE 3

### Single Strand Conformation Polymorphism (SSCP) of BYDV Replicase Sequence

Aliquots of the purified POLR and FSF amplified PCR products obtained via the procedure detailed in Example 2, were analysed by SSCP. Samples were heated at 95° C. for 5 min in the presence of 50% formamide. Sample was quenched on ice, mixed vol./vol. with loading buffer (95% formamide, 20 mM EDTA, Xylene-cyanol and bromophenol blue 500 mg/l), heated for 5 min at 95° C., cooled for 2 min on ice and loaded on to a 12% polyacrylamide gel (PAGE). A vertical PAGE Mini-Protean II apparatus was used (BioRad). The gel was run in a water bath at 15° C. for 2-4 h at 200-250V. The running buffer was 1×TBE (90 mM Tris-borate, 2 mM EDTA).

The PAGE gel was silver stained according to Bassam et al. (1991).

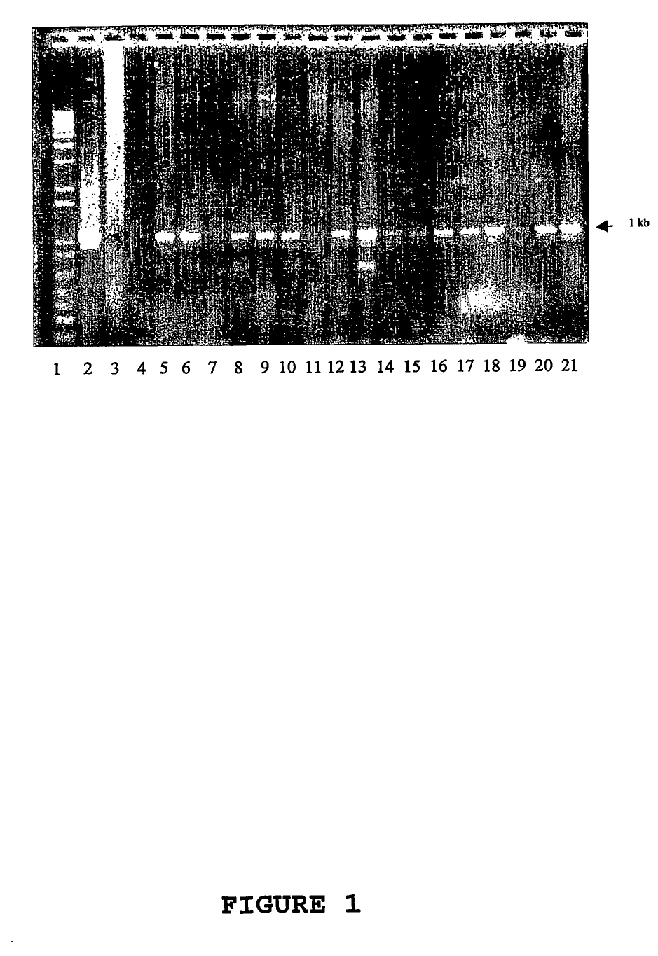

The SSCP patterns did not correlate with the virus serotype. The replicase sequence SSCP of the RPV and PAV serotypes was similar. ORF2 amplifying primers POLR and FSF produced SSCPs that fell into two groups, those that had one band and those with two bands. FIG. 1 shows the PCR products for Rep5 gene generated with primers Rep4 and Rep5 (1 kb). Lane 1 shows a 1 kb DNA marker; lane 2 shows a plasmid positive control; lane 3 shows wheat (c.v Westonia) untransformed control and lane 4 is a water control. Lanes 5, 6, 7, 8, 9, 10, 11, 12, 13, 14, 15, 16, 17, 18, 19, 20 and 21, show positive PCR results for transformed lines containing sense Rep5m gene, while lane 7 shows a negative PCR result.

In the group with two bands the samples 3, 7, 9 and 30 had an identical banding pattern. Samples 8, 11 and 14 were different and formed one group. Samples 10, 24, 26 and 27 formed a third group. Of the samples with one band only samples 1, 2, 4, 5, 16, 22 and 28 had identical SSCP patterns. In total 5 samples had a unique pattern (6, 13, 18, 20, 25).

Of the three samples (3, 7 and 16) two (3 and 7) had identical SSCP patterns. A similar pattern was produced by the Australian BYDV-PAV. The result agrees well with sequencing results in which the nucleotide sequences of samples 3, 7 and the Australian BYDV-PAV sequence differed only by 5-6%. Sample 16 with a different SSCP pattern differed in sequence by 10-12% (see Example 5 below).

### EXAMPLE 4

### Cloning of the Replicase Gene

Based on the SSCP results shown in Example 3 with the POLR and FSF primers, three samples were selected for replicase sequence cloning. Of the samples with single-banded SSCP results, the replicase of sample 16 was cloned. Of the samples with double-banded SSCP results, 3 and 7 were selected. The cloned sequence was 1608 bp long and corresponds to the BYDV-PAV nucleotides 1141-2749 (Miller et al. 1988a). The cloned fragments included the entire ORF2.

Purified PCR products for ORF2 from Example 2 was cloned into pCR 2.1-TOPO plasmid with TOPO-TA Cloning-kit (InVitrogen) following the manufacturer's instructions. Briefly, the 1600 bp long PCR products of the POLR and FSF primers were ligated into the vector by standard ligation procedures, and transformed into competent E. coli strains DH5α and/or TOP10-cells (Invitrogen) by standard heat-shock method. Transformed cells were allowed to grow for 30 min at 37° C. then plated on selective media and grown overnight at 37° C.

A selection of putative transformants were selected, and regrown for mini DNA preparation. A standard mini-preparation method was used, and the isolated DNA was then tested by restriction enzyme digestion/agarose gel electrophoresis. If the plasmid contained an insert the restriction product was two fragments of around 3900 and 1600 bp in size. All of the transformants produced the expected size fragments. The orientation of the insertion was also checked with BamHI restriction. The vector contains only one BamHI site, and based on the published sequence of BYDV-PAV there is only one BamHI site at 600^{th} nucleotide. Accordingly, if the replicase sequence was inserted in sense-orientation the 639 and 4877 bp fragments were produced. If the insert was antisense then the fragments were 1047 and 4469 bp. Of the tested samples 3 of 9 transformants had the replicase sequence in sense orientation and 5 in antisense orientation. One sample did not cut.

### EXAMPLE 5

### Sequencing of the Replicase Fragments

Sequencing was carried out using an automated sequencer ABI PRISM 377 (Perkin Elmer) in accordance with the manufacturer's instructions. The sequencing was performed with the following primers: M13 Forward (−20) (GTAAAACGACGGGCCAG), M13 Reverse (CAGGAAACAGCTATGAC), SEKV1 (TGAAATTCAACGAGAGAAGAA) and SEKV2 (AAAGCCATTGCATCCT). All the sequences were analysed with GCG-program (Wisconsin Package Version 8.1-unix Genetics Computer Group, Madison Wis.). In homology comparison used with Pile-Up program gap creation penalty was 5 and gap extension penalty was 0.3.

The replicase sequences from different field samples were very similar. The field samples 3 and 7 had 95% homology in the replicase sequence. Field sample 16 differed a little, however, the homology was as high as 88-89% with other samples. In length the cloned sequences were 1609 (sample 3), 1610 (sample 7) and 1612 (sample 16) base pairs.

The degree of variation from the published sequence of BYDV-PAV were similar in different samples. Samples 3 and 7 differed from the published BYDV-PAV sequence by 6% and sample 16 by 10%. Sequence differences extend evenly throughout the replicase sequence, although the applicant noted that the level of sequence divergence was not sufficient to adversely effect the RNA-mediated post transcription gene silencing.

### EXAMPLE 6

### Silencing Constructs

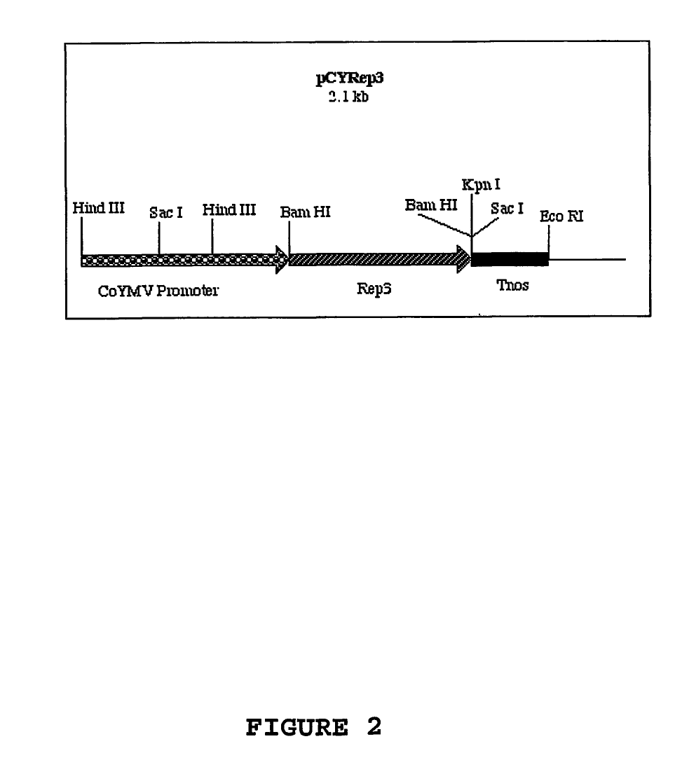

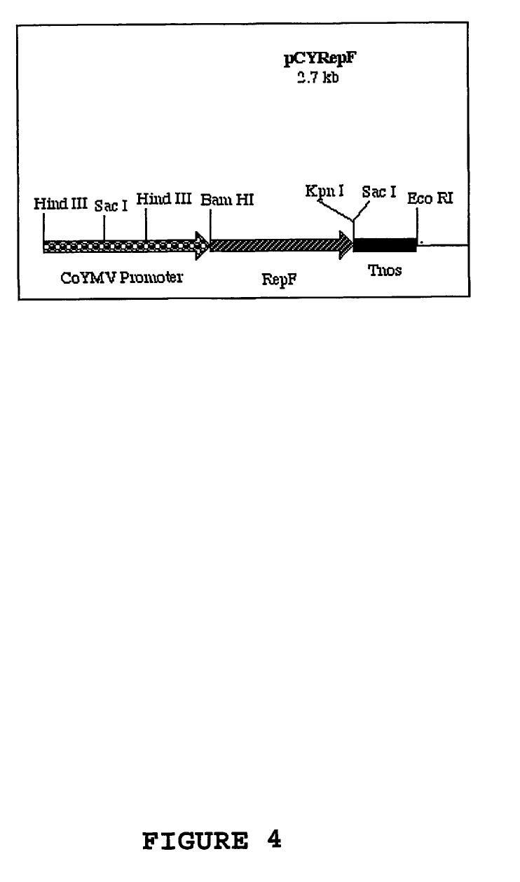

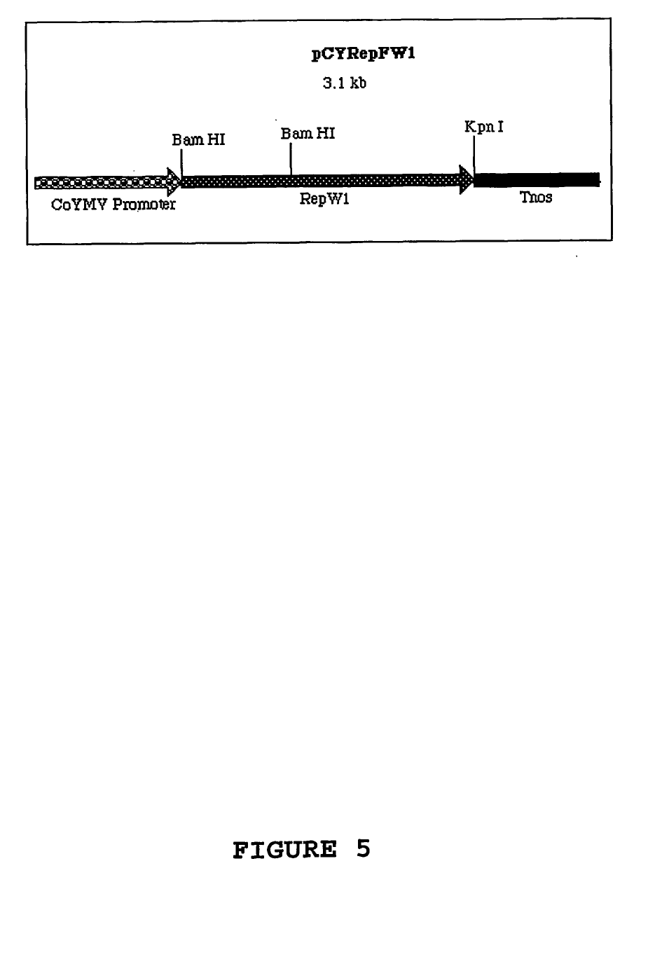

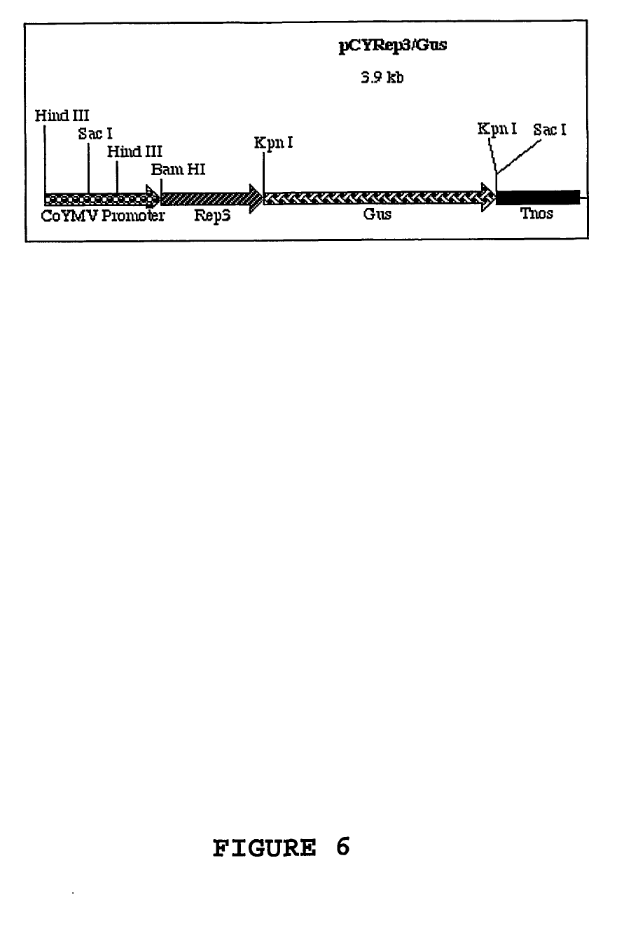

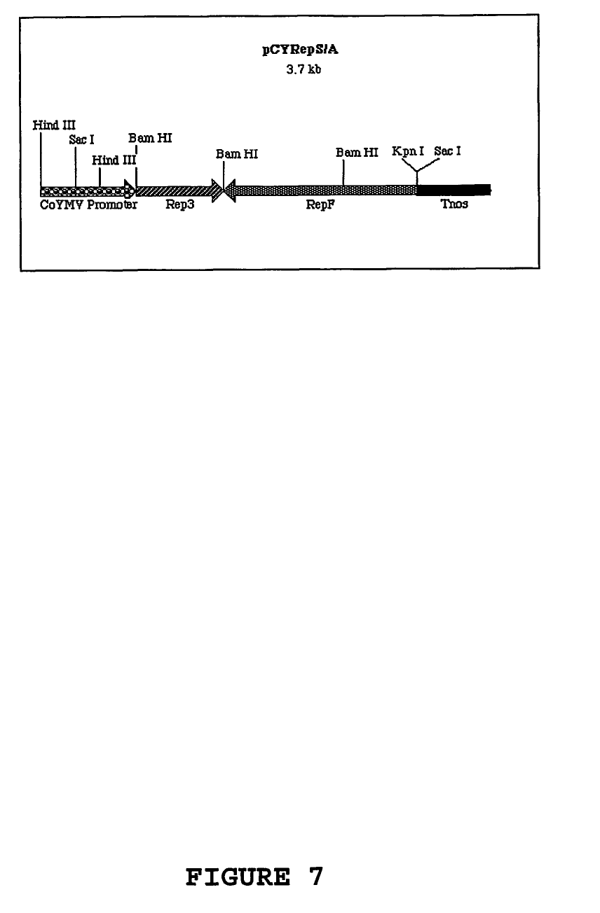

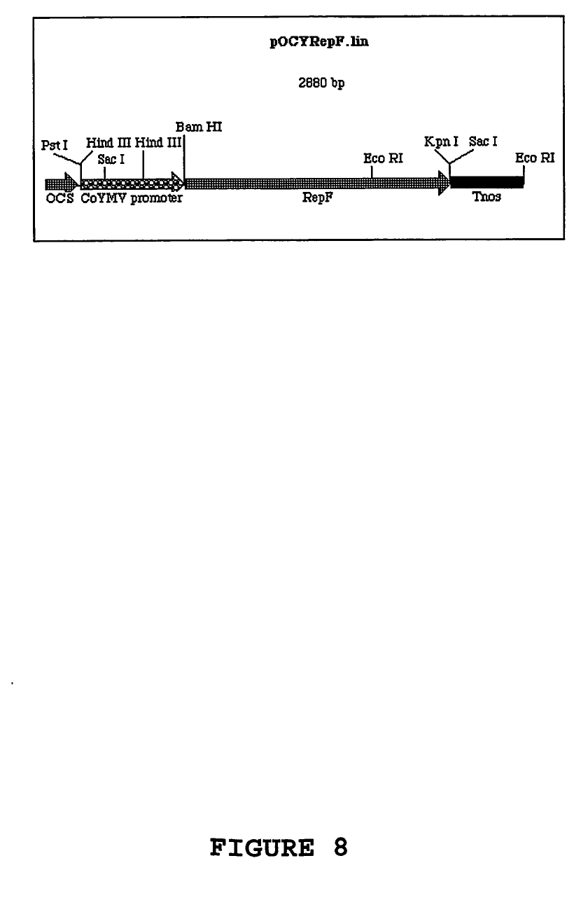

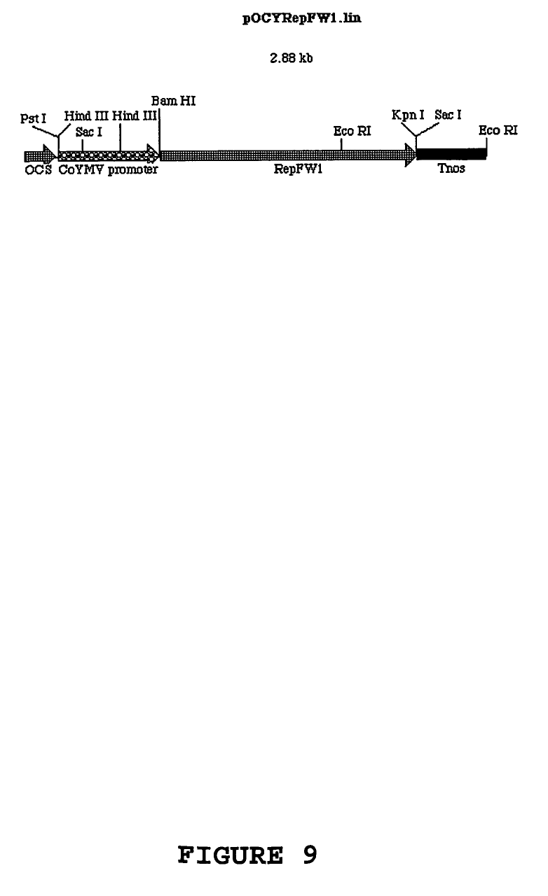

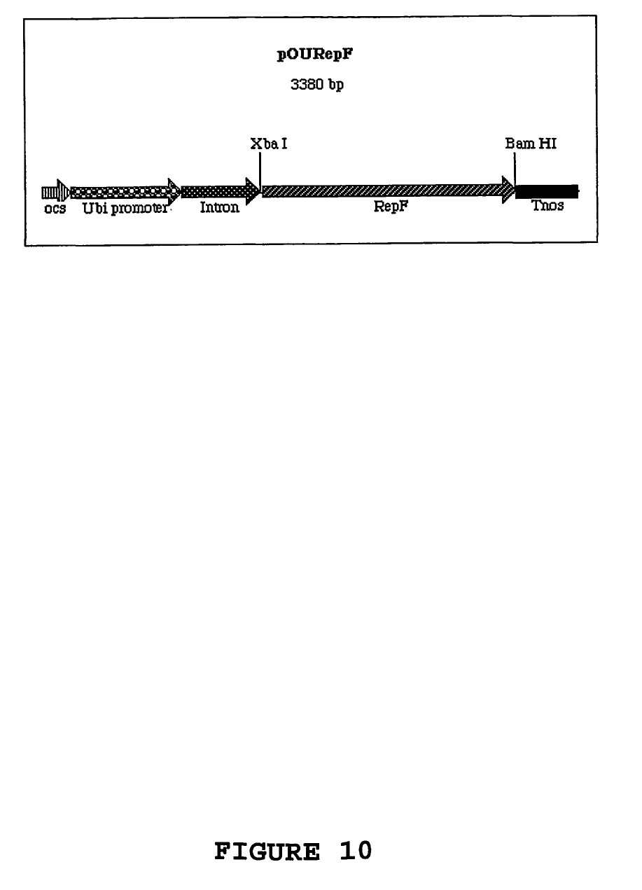

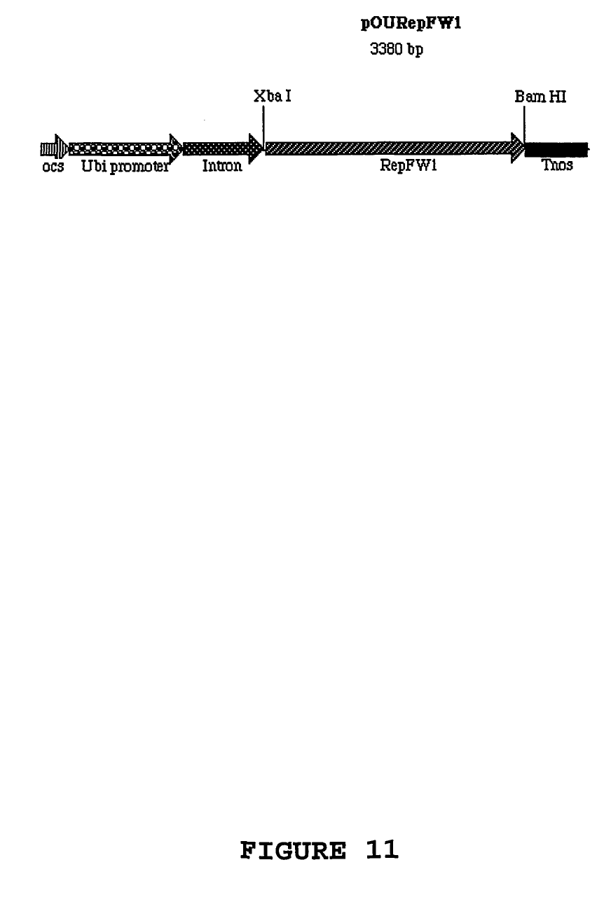

Ten constructs containing the replicase sequence driven by different promoters and in different configurations (eg full length, truncated, in a tandem sense and antisense configurations) were made, and these are shown in FIGS. 2 to 11.

### EXAMPLE 7

### Production of Transgenic Wheat Plants Containing Introduced DNA Constructs

A population of transgenic wheat plants, containing DNA constructs as discussed in Example 6, was generated using the following procedures.

Target Tissues

Wheat plants (cultivars Westonia, Brookton) were grown at 22-24° C. in a glasshouse. Seeds containing immature embryos were harvested at 11-15 days post-anthesis and surface sterilised. Immature embryos were excised and placed on MS (Murashige and Skoog, 1962) medium containing 2.5 mg/l 2,4-dichlorophenoxyacetic acid (2,4-D) for two days prior to bombardment.

Microprojectile Bombardment

Osmoticum treatments of target tissues, DNA precipitation and microprojectile bombardment were performed as described for sugarcane (Bower et al., 1996) with the exception of the use of tungsten particles. Wheat tissues were bombarded with 50 μg of gold particles per bombardment. The plasmids used for bombardment were pEmuKN (Last et al., 1991), which encodes neomycin phosphotransferase (NptII), in equimolar concentrations with constructs based on the BYDV replicase. In some experiments where the uida gene was included in the microprojectile precipitation the plasmid ratios for the NptII, replicase and uida gene constructs were adjusted to 2:1:1 respectively. The series of constructs to be tested for efficiency in conferring BYDV resistance is described in Table 3.

TABLE 3
BYDV constructs Used for Wheat Transformation

| | Construct | | Insert | |
|---|---|---|---|---|
| No | Name | Promoter | size-Kb | Comments |
| 1 | pCYRep3 | CoYMV | 1.0 | 5′ truncated ORF2 of |
| | | | | PAV-F isolate, |
| | | | | untranslatable. |
| 2 | pCYRep5 | CoYMV | 0.6 | 3′ truncated ORF2 of |
| | | | | PAV-F isolate, potentially |
| | | | | translatable 104 aa. |
| 3 | pCYRepF | CoYMV | 1.6 | Full-length ORF2 of |
| | | | | PAV-F isolate. |
| | | | | Potentially translatable |
| | | | | 438 aa. |
| 4 | pCYRepFW1 | CoYMV | 1.6 | Full-length ORF2 from |
| | | | | PAV-WA1. Potentially |
| | | | | translatable 438 aa. |
| 5 | pCYRepS/A | CoYMV | 2.6 | Full-length ORF2 of |
| | | | | PAV-F isolate inserted |
| | | | | downstream of Rep3 in |
| | | | | antisense orientation |
| | | | | in pCYRep3. |
| 6 | pCYRep3Gus | CoYMV | 2.8 | uidA gene inserted |
| | | | | downstream of Rep3 in |
| | | | | sense orientation in |
| | | | | pCYRep3. |
| 7 | pOCYRepF | CoYMV | 1.6 | An ocs enhancer located |
| | | | | upstream of CoYMV |
| | | | | promoter in pCYRepF. |
| | | | | Potentially translatable |
| | | | | 438 aa. |
| 8 | pCYRepFW1 | CoYMV | 1.6 | Full-length ORF2 from |
| | | | | PAV-WA1. Potentially |
| | | | | translatable 438 aa. |
| 9 | pOURepF | Ubi | 1.6 | Full-length ORF2 of |
| | | | | PAV-F isolate with ocs |
| | | | | enhancer upstream of ubi |
| | | | | promoter. Potentially |
| | | | | translatable 438 aa. |
| 10 | pOURepW1 | Ubi | 1.6 | Full-length ORF2 from |
| | | | | PAV-WA1 with ocs enhancer |
| | | | | upstream of ubi promoter. |
| | | | | Potentially translatable |
| | | | | 438 aa. |

Note:
CoYMV promoter = Commelina yellow mottle virus promoter (Medberry et al., 1992)
ocs = octopine synthase enhancer element (Ellis et al., 1987)
Ubi promoter = Maize ubiquitin promoter (Christensen and Quail, 1996)
uidA (Gus) gene = β-glucuronidase marker gene (Jefferson et al., 1986)
PAV-F = PAV Finland isolate
PAV-WA1 = PAV Western Australian isolate No 1
RepF = 1610 nucleotide (nt) fragment containing the full length of ORF 2 of the Finland PAV isolate.
RepFW1 = 1610 nt fragment containing the full length of ORF 2, PAV-WA1.
Rep3 = 1000 nt 3′ end of ORF 2 from the PAV-F isolate.
Rep5 = 5′ end 610 nt of ORF 2 from the PAV-F isolate.
All replicase gene constructs contain a nos terminator sequence (nos)

Constructs were designed and made to enable transformation of wheat plants with inserts of a structure that stimulates the post-transcriptional degradation of RNA mechanism, resulting in specific degradation of the BYDV replicase RNA and resistance to subsequent BYDV infection. The characteristics of such DNA integration structures are expected to result from the complex integration patterns of multiple inserts commonly associated with microprojectile bombardment (Bower et al., 1996), or from introduction of inserts containing tandem or inverted repeats of the BYDV replicase gene. The precise mechanisms of induction of post-transcriptional RNA degradation, and the degradative process have not been conclusively identified.

The portions of the replicase gene contained in the series of plasmids listed in Table 3 were potentially capable of producing a replicase protein truncated to different degrees, or in some cases were untranslatable. Our research aims to identify transgenic lines which contain the replicase gene, but which do not produce a significant amount of the replicase protein, due to post-transcriptional degradation of the replicase RNA by the plant. The capacity of the plants to degrade specific RNA species by this mechanism can be expected to confer resistance to BYDV in wheat and other cereals.

Selection Procedures

Following bombardment the embryos were placed on MS medium containing 2.5 mg/l 2,4-D for two weeks at 24° C. in the dark, transferred to the same medium plus 150 mg/l kanamycin (Sigma) for a further two weeks under the same culture conditions. The tissues were then transferred to MS medium containing 0.1 mg/l 2,4-D and 150 mg/l kanamycin, maintained in the dark for two days, then placed in the light for regeneration. After two weeks tissues were transferred to the same medium, but lacking 2,4-D. Green transgenic (Tₒ) plants were transferred to ½ strength MS to produce roots and then established in pots in the glasshouse.

### EXAMPLE 8

### Challenges of Transgenic Wheat Lines with BYDV

In order to determine whether or not the introduced constructs were capable of protecting wheat plants from BYDV infection a number of experiments were carried out.

Detection of Transgenes in wheat lines

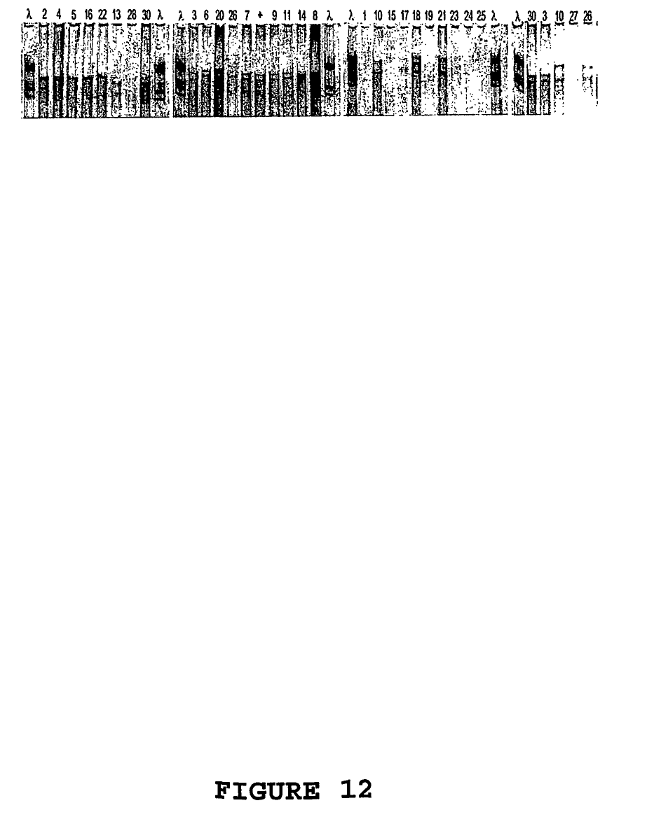

A population of T₀ lines resistant to kanamycin was generated, as described above, and plants were analysed by PCR to determine which of the three genes used in the transformation experiments were stably integrated in the genomes of these lines. The results of analysis of the independently transformed T₀ plants containing the replicase gene are summarised in Table 4 and the results of a PCR test for the presence of the introduced BYDV replicase gene is shown in FIG. 12.

TABLE 4
Transgenic Wheat Lines Selected for Challenge with BYDV

| | | | | Evidence |
|---|---|---|---|---|
| | | Replicase | Other | for |
| Line | Cultivar | gene | genes | Rep gene |
| W269-1 | Westonia | Rep3¹ | Npt II, | |
| | | | uidA | lane 5 |
| W269-2 | Westonia | Rep3 | Npt II, | |
| | | | uidA | lane 6 |
| W269-3 | Westonia | Rep3 | Npt II, | |
| | | | uidA | lane 8 |
| W275-2b | Westonia | Rep3 | Npt II, | |
| | | | uidA. | lane 9 |
| W275-2a | Westonia | Rep3 | Npt II, | |
| | | | uidA | lane 10 |
| W275-3a | Westonia | Rep3 | Npt II, | |
| | | | uidA | lane 12 |
| W275-3b | Westonia | Rep3 | Npt II, | |
| | | | uidA | lane 13 |
| W275-5 | Westonia | Rep3 | Npt II, | |
| | | | uidA, | lane 14 |
| W275-8 | Westonia | Rep3 | Npt II, | |
| | | | uidA | lane 16 |
| B265-2 | Brookton | Rep3 | Npt II, | |
| | | | uidA | lane 17 |
| B273-1 | Brookton | Rep3 | Npt II, | FIG. 2, ' |
| | | | uidA | lane 18 |
| B273-5 | Brookton | Rep3 | Npt II, | |
| | | | uidA | lane 20 |

¹RepS = untranslatable 5′ truncated replicase gene in pCY Rep3

DNA for PCR analysis was isolated using the following procedure. A 2 cm long section of the newest fully expanded leaf was harvested into a microfuge tube. After leaf tissue was ground in liquid nitrogen, 800 μl of extraction buffer (0.1M Tris, pH8, 50 mM EDTA, pH8, 0.5M NaCl, 1.3% SDS, 0.3% β-mercaptoethanol) was added. Samples were incubated at 65° C. for 20 min with gentle mixing at 5 min intervals. After addition of cold 5 Mpotassium acetate and incubation on ice for 5 min the samples were centrifuged and the supernatant transferred to another microfuge tube. Genomic DNA was precipitated by addition of isopropanol, resuspended in 20 μl water and used for PCR analysis.

The presence or absence of the NptII, uidA and BYDV replicase genes in the transgenic lines was tested using the following primers:

NptII - 5′Kan
5′GCTTGGGTGGAGAGGCTATTC-3′
3′Kan
5′-ATCACGGGTAGCCAACGCTAT-3′
uidA - 5′GUS
5′-CGGGGTACCCCGATGTTACGTCCTGTAG-3′
3′GUS
5′-GGGTACCCCTCATTGTTTGCCTCCCTGC-3′
Replicase - Rep4
5′-GATCCCCACTGTGGCT-3′
Rep5
5′-GGTAATTAATATTCG-3′

PCR reagents were supplied by Perkin-Elmer (AmpliTaq® DNA Polymerase, 10×PCR Buffer II [500 mM KCl, 100 mM Tris-HCl pH8.3], 25 mM MgCl₂), Promega (deoxynucleoside triphosphates (dNTPs)) and Life-Technologies (PCR primers). PCR reactions consisted of 1 μl template, 10 pmol of each primer, 4 mM MgCl₂, 1×PCR Buffer II, 10 mM dNTPs, 1.25 U AmpliTaq DNA Polymerase in 50 μl total volume. PCR cycling conditions consisted of an initial denaturation period of 3 min at 94° C. followed by 30 cycles of 94° C. 30 sec, 60° C. 30 sec, 72° C. 2 min, followed by a final extension cycle of 72° C. for 7 min. Reactions were performed in a Perkin Elmer PCR System 2400 Thermal Cycler.

Negative controls consisted of leaf tissue from a non-transgenic Westonia wheat plant and a PCR reaction using water as a sample template. Positive controls consisted of 20 ng of the appropriate plasmid DNA and, in the case of the uidA gene, of leaf tissue from a plant known to contain that gene.

PCR reactions were run on an agarose gel and stained with ethidium bromide to enable visualisation of bands corresponding to the fragment size predicted from the sequence information and from the positive control reaction.

BYDV Challenges

To determine whether a proportion of plants containing the replicase-based constructs show resistance to BYDV infection, as predicted on the basis of the method of introduction of the constructs and on the design of the constructs, the transgenic lines were analysed using the following procedures.

From the subpopulation of T₀ wheat plants (cvs Westonia, Brookton) that were found to contain the introduced replicase gene 12 plant lines were selected for further analysis, and a T₁ population was generated for each of these lines. Each T₁ population consisted of 15 plants and these were tested for resistance to BYDV using the challenge protocol described below.

BYDV Infection

Fifteen T₁ seeds from each line were planted and grown to a three leaf stage for challenge with BYDV-PAV (WAl isolate). In addition, ten non-transgenic lines of cultivar Westonia were grown and infected in parallel to confirm the efficiency of the BYDV infection procedure. The virus was maintained in wheat and paspalum plants grown in a growth chamber at 18° C. A colony of the oat aphid (Rhopalosiphum padi), an efficient vector for spread of BYDV in wheat, was maintained on wheat plants grown in aphid cages.

For each wheat plant to be challenged, 10 aphids in the early non-winged stage of development were collected and stored in a Petri dish for 3-8 hrs before incubation with BYDV infected leaves. The leaves were prepared in the following manner. Young leaves from BYDV infected wheat plants grown at 18° C. were sliced from the plant in the late afternoon and placed with their cut ends in MS medium. The aphids and leaves were co-incubated overnight at 18° C. to ensure the aphids were able to act as highly effective vectors for the virus. The next day, 10 of these aphids were placed on each plant to be challenged and a plastic container placed over the plant to contain the aphids. The plants and aphids were co-incubated at 18° C. for 24 hrs, then the aphids were killed and the plants returned to a 20° C. controlled environment chamber for three weeks to enable BYDV infection to develop in susceptible plants.

Detection of BYDV in Plants

To determine whether the transgenic T₁ lines that were inoculated with BYDV, as described, were resistant or susceptible to BYDV infection, Enzyme Linked Immuno-Sorbant Assays (ELISAs) were performed on leaf tissues from the newest fully expanded leaf of the 15 T₁ progeny of each of the original transgenic T₀ lines. Positive controls for the ELISA assays consisted of leaf tissue from previously infected wheat plants and negative controls consisted of leaf tissue from uninfected Westonia plants. Leaf tissue from 10 non-transgenic Westonia wheat plants, plus all T₁ null segregants for the replicase transgene from each T₀ plant, infected in parallel with the transgenic population were assayed to confirm the effectiveness of the BYDV challenge protocol. The ELISA assay was performed using a PLANTEST ELISA kit (PHYTO-DIAGNOSTICS) that detects the presence of the BYDV coat protein. All samples were assayed in duplicate. The resistance, or susceptibility, of each of the 15 T₁ plants from each original T₀ plant line was assessed by comparison with the readings from the ELISA assay of BYDV infected wheat plants and non-infected plants. Summarised results of these data are shown in Table 5

TABLE 5
Summary of Resistant and Susceptible Phenotypes in Wheat
Lines After Infection with BYDV

| | | | No. | | | |
|---|---|---|---|---|---|---|
| | | No. T₁ | suscep- | No. | No. | No. |
| | No. T₁ | Rep +ve | tible | resistant | delay | recovery |
| Tₒ Line | Plants | plants¹ | plants | plants² | plants³ | plants⁴ |
| W269-1-1 | 14 | 8 | 6 | 2 | | |
| W269-2-1 | 15 | 8 | 7 | 1 | | |
| W269-3-1 | 15 | 8 | 4 | | 3 | 1 |
| W275-2a-1 | 15 | 10 | 9 | | 1 | |
| W275-2b-1 | 15 | 9 | 5 | 2 | 2 | |
| W275-3-1 | 15 | 10 | 5 | 1 | 3 | 1 |
| W275-3b-1 | 15 | 13 | 10 | 1 | 1 | 1 |
| W275-5-1 | 15 | 14 | 9 | 1 | 3 | 1 |
| W275-8-1 | 11 | 6 | 3 | 1 | 1 | 1 |
| B265-2-1 | 15 | 13 | 5 | | 5 | 3 |
| B273-1-1 | 15 | 13 | 6 | 4 | 2 | 1 |
| B273-5-1 | 15 | 7 | 5 | 1 | 1 | |

¹Plants that tested positive in PCR assays to detect the replicase sequence
²Plants that tested negative in both ELISAs for BYDV coat protein
³Plants that tested negative in the first ELISA but positive in the second
⁴Plants that tested positive in the first ELISA but negative in the second

Because the T₀ plants were expected to give rise to a segregating population in the T₁ generation, PCR assays to detect the presence or absence of the replicase gene were performed on each of the challenged plants (Table 3, FIG. 1).

Lane 1 shows a 1 kb DNA marker, while lane 2 shows a plasmid positive control. Lane 3 shows an untransformed wheat (c.v Westonia) control and lane 4 is a H₂O control. Lanes 5, 6, 7, 8, 9, 10, 11, 12, 13, 14, 15, 16, 17, 18, 19, 20 and 21, are transformed lines containing sense Rep5m gene, and show positive PCR results. Lane 7 shows a negative PCR results.

Following inoculation with BYDV, as described, plants were grown for three weeks before leaf samples were taken for ELISA assays to detect the presence of the virus in infected leaf tissue. The ELISAs were repeated at six weeks after germination to determine the level of stability of resistance to BYDV in lines showing low ELISA readings. All data points were calculated by subtraction of the ELISA reading, from a non-transgenic, uninfected Westonia wheat plant of the same age as the infected plant, and measured on the same plate. Subtraction of this value resulted in slightly negative values in some plants showing resistance to viral infection. Plants that showed ELISA values of less than ⅕th the value of the positive control value were rated as ELISA negative, indicating resistance to BYDV infection or delayed development of viral infection.

Of 175 T₁ progeny tested from 12 independent transgenic wheat lines, 119 plants were assayed as PCR positive for the introduced replicase gene and of these 14 plants gave low ELISA readings in two consecutive assays showing that BYDV coat protein levels were either very low or absent in the leaf tissue. Many of these ELISA readings were very low, indicating high levels of resistance. A further 22 plants showed delayed onset of disease as evidenced by low ELISA readings for the initial test followed by increased readings in the second ELISA. Nine plants (Table 5) showed a recovery phenotype that is consistent with initial development of BYDV infection, as expected from the high levels of inoculation used, followed by a failure of the virus to replicate to levels required to sustain the infective process.

These data support our belief that the challenge and ELISA protcols are very reliable because 100% infection rates in non-transgenic controls were obtained, and all 56 null segregants (PCR negative lines) showed ELISA positive results by the second assay.

These results show that it is possible to produce BYDV resistant wheat plants by non-protein-mediated mechanisms resulting from introduction of replicase gene sequences.

### EXAMPLE 9

### BYDV Resistant Plant Lines

Plant lines showing resistance to BYDV were expected to do so as a result of post-transcriptionally mediated gene silencing mechanisms, as opposed to protein mediated mechanisms, because the replicase sequence in that construct should be untranslable due to truncation of the 51 portion of the gene. However, in order to confirm that the transgenic BYDV resistant plant lines were resistant because of post-transcriptionally mediated gene silencing mechanisms, a reverse transcriptase-PCR (RT-PCR) assay was used to estimate BYDV replicase mRNA levels in leaves.

Plants containing the replicase gene driven by the construct, pCYRep5, and showing a positive signal for replicase RNA in the RT-PCR assay were expected to be susceptible to BYDV, unless the resistance mechanism was protein mediated. If the resistance was conferred by a post-transcriptionally mediated gene silencing mechanism no signal corresponding to the presence of the replicase mRNA should be present in resistant lines. Although the absence of a replicase mRNA signal could also result from transcriptional silencing of the introduced gene, these lines could not be resistant due to protein mediated resistance mechanisms due to the lack of transcription from the introduced genomic replicase sequence. Thus an absence of a RT-PCR signal (in association with a positive control actin signal) for the replicase gene fragment, in resistant lines, would indicate that the resistance mechanism was not protein mediated.

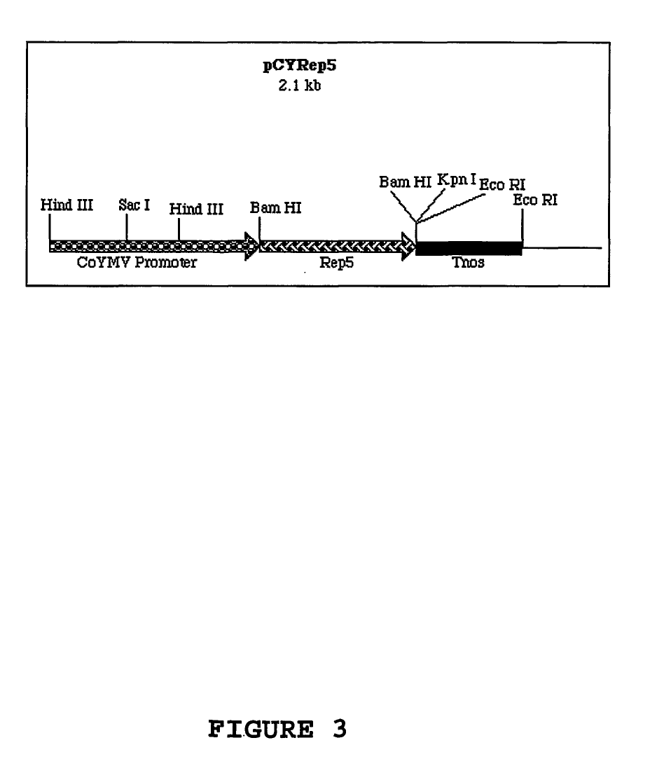

Three resistant lines (W269-1-1-11, W269-1-1-12 and W269-2-1-12) which contained the pCYRep5 construct as discussed in Example 6 and shown in Table 3 and FIG. 3 and one susceptible line (W269-1-4-9) were selected from Population I, T₂ generation fbr RT-PCR assays. Non-transformed plants (c.v. Westonia) were used as negative controls. An actin positive control was included to confirm that the RNA extractions and PCR conditions were effective. This produced a band of approximately 480 bp.

Seeds were sown in the PC2 glasshouse.

Leaf material for RNA extraction was harvested at 2-leaf stage and RNA was extracted with RNAqueous plus Plant RNA Isolation Aid kit (Geneworks) in accordance with the manufacturer's instructions.

RT-PCR reagents were supplied by Applied

Biosystems (Perkin-Elmer): MuLV Reverse Transcriptase, RNase Inhibitor, Golden AmpliTaq DNA polymerase, 10×PCR buffer II (500 mM KCl, 100 mM Tris-HCl pH 8.3, and 25 nM MgCl₂).

Primers Rep6, Rep7, ActF and ActR were Synthesised by Invitrogen (Life-Technologies)

| | |
|---|---|
| Rep6: | 5′-AAGGTGAGAGGACACAGAATGTCC |
| Rep7: | 3′-GGTATGGAAAGCAGTATTG |
| ActF: | 5′-ACC TGATGAAGATCCTCAC |
| ActR: | 3′-TCCTCCAATCCAGACAC |

RT-PCR Conditions

The RT-PCR conditions were as follows. Buffers consisted of 4 mM MgCl₂, 1×PCR Buffer II, 5 U RNase Inhibitor, 12.5 U MuLV Reverse Transcriptase, 1 mM dNTPs, 10 pmol of each downstream primer (Rep7 or ActR:3′), 1 μl RNA, and DEPC-treated H₂O to a final volume of 10 μl. The reaction was incubated at 42° C. for 15 min and then 96° C. for 5 min to denature the reverse transcriptase enzyme. For PCR, the volume was increased to 20 μl maintaining conditions of 4 mM MgCl₂ and 1×PCR Buffer II, and adding 2 U Tag DNA polymerase and 10 pmol of each upstream primer (Rep6 or ActF:5′). The PCR cycling run consisted of an initial denaturation period of 5 min at 94° C. followed by 30 cycles of 94° C. 30 sec, 58° C. 30 sec, 72° C. 1 min, followed by a final extension cycle of 72° C. for 10 min.

A separate PCR reaction was performed for each sample in which the RT PCR step was omitted to confirm that the bands observed were not due to contamination with residual DNA. No bands were observed from any of the samples (data not shown) confirming that no DNA contamination was present. PCR reactions were run in agarose gels under standard conditions and bands were visualised using ethidium bromide staining.

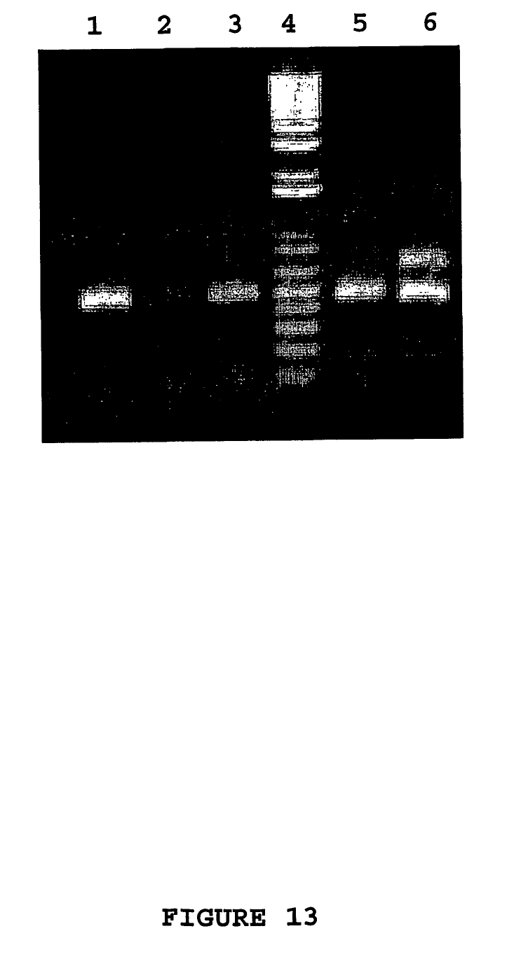

FIG. 13 shows the results of RT-PCR assays for the BYDV-PAV replicase mRNA in wheat plants. Lanes 1-3 shows that BYDV resistant plants have a positive actin band, but no replicase band. Lane 4 shows a 1 Kb DNA molecular marker, while lane 5 shows a non-transgenic control (Westonia) showing only an actin band. Lane 6 shows a BYDV susceptible transgenic plant with both the replicase and actin gene bands.

The susceptible plant (Lane 6) showed a strong band from the replicase mRNA showing that expression of the replicase gene fragment in pCYRep5 did not result in resistance in that line. It also confirmed that the RT-PCR assay amplified from the replicase mRNA effectively. The RT-PCR assays on the three BYDV resistant lines showed a band generated by the actin gene primers, but no band from the replicase gene primers, indicating that the replicase mRNA was either absent in the sample or present at very low levels. This confirmed that the resistance observed in these lines was not protein mediated and was attributable to a mRNA mediated, post transcriptional gene silencing mechanism.

### REFERENCES

Anderson J M, Bucholtz D L, Greene A E, Francki M G, Gray S M, Sharma H, Ohm H W and Perry K L, 1998. Characterisation of wheatgrass-derived barley yellow dwarf virus resistance in a wheat alien chromosome substitution line. Phytopathology 88: 851-855

Audy P, Palukatis P, Slack S A and Zaitlin M, 1994. Replicase-mediated resistance to Potato virus Y in transgenic tobacco plants. Molecular Plant-Microbe Interactions 7: 15-22

Bower, R., Elliott, A. R., Potier, B. A. M. & Birch, R. G. High-efficiency, microprojectile-mediated cotransformation of sugarcane, using visible or selectable markers. Molecular Breeding, 2, 239-249.

Brederode F T H, Taschner P E M, Posthumus E and Bol J F, 1995. Replicase-mediated resistance to alfalfa mosaic virus. Virology 207: 467-474

Carr J P, Marsh L E, Lomonossoff G P, Sekiya M E and Zaitlin M, 1992. Resistance to tobacco mosaic virus induced by the 54-kDa gene sequence requires expression of the 54-kDa protein. Molecular Plant-Microbe Interactions 5: 397-404

Christensen, A. H. and Quail, P. H (1996). Ubiquitin promoter-based vectors for high-level expression of selectable and/or screenable marker genes in monocotyledonous plants.

Transgenic Res 5, 1-6.

Cooper and Jones, 1983. Responses of Plants to Viruses: Proposals for the Use of Terms. Phytopathology 73(2): 127-128.

Ellis et al. 1987. EMBO J. 6:11-16

Francki M G, Crasta O, Anderson J M, Sharma H and Ohm H W, 1997. Structural organisation of an alien Thinopyrum intermedium group 7 chromosome in US soft red winter wheat (Triticum aestivum L.). Genome 40: 716-722

Guo H S, Cervera M T and Garcia J A, 1998. Plum pox potyvirus resistance associated to transgene silencing that can be stabilised after different number of plant generations. Gene 206: 263-272

Hewings A D and Eastman C E, 1995. Epidemiology of barley yellow dwarf in North America, Pages 75-106 in Barley Yellow Dwarf Virus: Forty Years of Progress. C J D'Arcy and P A Burnett, Eds. The American Phytopathological Society, St Paul, Minn.

Jefferson, R. A. et.al., Proc. Natl. Acad. Sci., 83, 8447-8451 (1986).

Jones A L, Johansen I E, Bean S J, Bach I and Maule A J, 1998. Specificity of resistance to pea seed-borne mosaic potyvirus in transgenic peas expressing the viral replicase (Nlb) gene. J. General Virology 79: 3129-3137

Kim C-H and Palukaitis P, 1997. The plant defence response to cucumber mosaic virus in cowpea is elicited by the viral polymerase gene and affects virus accumulation in single cells. EMBO J. 16: 4060-4068

Last et al., 1991

Lister R M and Ranieri R, 1995. Distribution and importance of barley yellow dwarf virus. Pages 29-53 in Barley Yellow Dwarf Virus: Forty Years of Progress. C J D'Arcy and P A Burnett, eds. The American Phytopathological Society, St Paul, Minn.

McElroy D, Blowers A D Jenes B and Wu R (1991). 1(act1) 5′ region for use in monocot transformation. Construction of expression vectors based on the rice actin. Molecular and General Genetics. 231: 150-160.

McKirdy S J and Jones R A C, 1996. Use of imidacloloprid and newer generation synthetic pyrethroids to control the spread of barley yellow dwarf luteovirus in cereals. Plant Dis. 80: 895-901

Medberry S L, Lockhart B E L and Olszewskine (1992). The Plant Cell 1992, 4: 185-192.

Miller W A and Rasochova L, 1997. Barley yellow dwarf viruses. Annu. Rev. Phytopathol. 35: 167-190

Miller W A, Waterhouse P M and Gerlach W L, 1988a. Sequence and organisation of barley yellow dwarf virus genomic RNA. Nucleic Acid Res. 16: 6097-6111.

Murashige, T. and F. Skoog. 1962. A revised medium for rapid growth and bioassays with tobacco tissue cultures. Physiol. Plant. 15:473-497.

Palukaitis P and Zaitlin M, 1997. Replicase-mediated resistance to plant virus disease. Advances in Virus Research 48: 349-377

Rubino L and Russo M, 1995. Characterisation of resistance to cymbidium ringspot virus in transgenic plants containing a full-length viral replicase gene. Virology 212: 240-243

Tenllado F, Garcia-Luque M, Serra M T and Diaz-Ruiz J R, 1995. Nicotiana benthamiana plants transformed with the 54-kDa region of the pepper mild mottle tobamovirus replicase gene exhibit two types of resistance responses against viral infection. Virology 211: 170-183

The claims defining the invention are as follows:

1. A method for protecting a plant from BYDV infection, comprising the step of transforming a modified nucleic acid molecule into a plant cell, wherein the expression of said nucleic acid molecule results in the expression of a translationally-altered RNA molecule which confers to said plant resistance against infection with BYDV.

2. A method according to claim 1, wherein the modified nucleic acid molecule is either cDNA, RNA, or a hybrid molecule thereof or a biologically active fragment thereof.

3. A method according to claim 1, wherein the nucleic acid molecule is a cDNA molecule encoding a replicase.

4. A method according to claim 3, wherein the replicase is a BYDV replicase.

5. A method according to any one of claims 1 to 4, in which the nucleic acid sequence comprises a nucleotide sequence corresponding to the sequence given in SEQ ID No. 1 or to the nucleotide sequence of a naturally occurring variant thereof, in which, compared to the sequence of SEQ ID No. 1 and/or the naturally occurring variant thereof, one or more nucleotides have been modified by addition, replacement and/or removal such that said nucleic acid molecule sequence is capable, upon transformation into a plant expressing a translationally-altered RNA molecule which confers to said plant resistance against infection with BYDV.

6. A method according to any one of claims 1 to 5, wherein the nucleic acid molecule is further modified so that a truncated translationally-altered RNA molecule is produced upon expression.

7. A method according to any one of claims 1 to 6, further comprising at least one step of cultivating the transformed plant cell into a mature plant.

8. A method according to any one of claims 1 to 7, further comprising at least one step of sexually or asexually reproducing or multiplying the transformed plant and/or the mature plant obtained from a transformed plant cell according to claim 7.

9. A method according to any one of claims 1 to 8, in which the plant is a plant that is susceptible to infection with BYDV.

10. A method according to claim 9, wherein the plant is a monocot.

11. A method according to claim 9 or claim 10, wherein the plant is selected from the group consisting of wheat (Triticum), sorghum (Sorghum), rice (Oryza), barley (Hordeum), maize (Zea), rye (Secale), triticale and oat (Avena).

12. A method according to claim 10, wherein the plant is wheat.

13. A genetic construct suitable for transforming a plant, said construct at least comprising a modified nucleic acid molecule, wherein the expression of said nucleic acid molecule results in the expression of a translationally-altered RNA molecule which confers to said plant resistance against infection with BYDV.

14. A genetic construct according to claim 13, at least comprising a nucleotide sequence that in which the nucleic acid sequence comprises a nucleotide sequence corresponding to the sequence given in SEQ ID No. 1 or to the nucleotide sequence of a naturally occurring variant thereof, in which-compared to the sequence of SEQ ID No. 1 and/or the naturally occurring variant thereof one or more nucleotides have been modified by addition, replacement and/or removal such that said nucleic acid molecule sequence is capable, upon transformation into a plant expressing a translationally-altered RNA molecule which confers to said plant resistance against infection with BYDV.

15. A genetic construct according to claim 13, wherein the nucleic acid comprises either:
a) a nucleotide sequence as shown in SEQ ID NO:1; or
b) a biologically active fragment of the sequence in a); or
c) a nucleic acid molecule which has at least 75% sequence homology to the sequence in a) or b); or
d) a nucleic acid molecule which is capable of hybridizing to the sequence in a) or b) under stringent conditions.

16. A genetic construct according to any one of claims 13 to 15, in which the nucleic acid sequence is under control of either the 35SCaMV promoter, CoYMV promoter, rice actin promoter, pEMU promoter, MAS promoter, maize H3 histone or maize ubiquitin promoter.

17. A genetic construct according to any one of claims 13 to 16, in a form that can be stably maintained or inherited in a micro-organism.

18. A genetic construct according to claim 17, wherein the micro-organism is a bacterium.

19. A genetic construct according to claim 18, wherein the bacterium can be used to transform a plant or plant material.

20. A genetic construct according to claim 19, wherein the bacterium is an Agrobacterium.

21. A micro-organism that contains a genetic construct according to any of claims 13 to 20.

22. A transgenic plant or plant cell, obtained by a method according to one of claims 1 to 12, or a progeny of such a plant.

23. A plant, plant cell, seed or plant material that has been transformed with genetic construct according to any one of claims 13 to 20, or a progeny of such a plant.

24. A plant according to claim 22 or claim 23, being a plant that is susceptible to infection with BYDV.

25. Cultivation material such as seed, tubers, roots, stalks, seedlings for a plant according to any one of claims 16 to 18.


SEQUENCE LISTING (1 sequence)

SEQ ID NO: 1 - DNA, 1610 bp, Barley yellow dwarf virus
tcttgactctgtgggtttttagaggggctctgtaccgcctctggttttgagagcccattccctattctcgggttgccagagattgcggtcacagacggagcccgactccgtaaggttagtagtaatattagataccttagccaaacccacctaggccttgtatataaggcaccaaatgcctccctgcacaacgcgcttgtggcagtggagagaagagtttttacagtaggaaagggggacaaagcaatctaccccccccgccctgagcatgacattttcactgatacgatggattatttccaaaaatccattatagaagaggtgggatactgtagaacatatccagcgcaactcctggctgacagctatagcgcaggaaagagggccatgtatcacaaagccattgcatccttgaagactgtcccttatcaccagaaggatgccaatgtgcaggctttcctgaagaaggaaaaacattggatgaccaaggacatcgccccccgattgatttgcccccgcagcaagcggtacaacatcatcctaggaactcgtttgaaattcaacgagaagaagatcatgcacgctatcgatagtgtgtctggatcccccactgtgctttctggctatgacaacttcaaacaaggaagaatcatagccaagaagtggcaaaagtttgtttgccccgtcgccatcggcgtggatgctagccgctttgaccaacacgtgtcagagcaggcgcttaagtgggaacacgggatatacaatgggatcttcggagacagcgaactggctcttgcacttgaacaccagatcaccaacaatatcaaaatgtttgttgaggacaagatgctcagatttaaggtgagaggacacagaatgtccggagacattaataccagcatgggaaacaaactgataatgtgtggcatgatgcatgcatatttcaagaagctgggtgttgaagctgagctatgcaataatggagatgattgtgtcatcataactgatagagtgaatgaagaacttttcagtggaatgtatgaccatttcctacaatacggcttcaacatggtgaccgagaagccagtttacgaactggaacaactggagttttgccagtcaaaaccggtctctattaatggaaagtatagaatggttagaaggcccgatagcataggcaaagatagcacaacactactgagcatgctcaaccaatccgacgtcaagagctatatgtcggctgtggctcagtgtggtttagtgctaaacgctggagtacccatacttgaaagtttctataaatgcctatatagaagctcggggtacaagaaagtgagtgaggaattcatcaaaaacgtcatttcgtatggaacagatgagagactacaaggtagacgtacctataatgaaacacctatcacaaaccacagtagaatgtcctactgggaatcattcggagttgaccctaagatacaacaaatcgtcgagaggtactacgacggtcttacggtaagtgcccaactccagagtgtgaaggtgacgactccacatctgcgatcaatactgctttccataccggaaaaccactcacaaaacgaatattaattacc